(19) United States
(12) Patent Application Publication
(10) Pub. No.: US 2003/0125528 A1
(43) Pub. Date: Jul. 3, 2003

(54) Process for preparing schiff base adducts of amines with o-hydroxy aldehydes and compositions of matter based thereon

(76) Inventors: Bruce A. Hay, East Lyme, CT (US); Michael T. Clark, Gales Ferry, CT (US)

Correspondence Address: PFIZER INC., PATENT DEPARTMENT, MS8260-1611, EASTERN POINT ROAD, GROTON, CT, 06340, US

(21) Appl. No.: 10/256,482

(22) Filed: Sep. 27, 2002

Related U.S. Application Data
(63) Continuation of application No. 09/337,985, filed on Jun. 22, 1999, now abandoned.
(60) Provisional application No. 60/090,714, filed on Jun. 26, 1998.

(51) Int. Cl. C07K 14/00

(52) U.S. Cl. 530/409; 530/410

(57) ABSTRACT

An improved process is described for preparing Schiff base condensation adduct final products whose components comprise a protein having beneficial activity in animals, and an aromatic o-hydroxy aldehyde, which comprises bringing together the above-mentioned components in an aqueous environment at a pH of 7.0 or higher to form a reaction mixture, under conditions effective to drive said condensation reaction substantially to completion by removing from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants. Preferred aromatic o-hydroxy aldehydes comprise o-vanillin; salicylaldehyde; 2,3-dihydroxybenzaldehyde; 2,6-dihydroxybenz-aldehyde; 2-hydroxy-3-ethoxybenzaldehyde; or pyridoxal. A very wide range of proteins may be employed. The improved process provides yields over 90% and substantially quantitative conversion of the aldehyde and protein to the condensation adduct.

The present invention is in the technical field relating to the synthesis of organic molecules comprising Schiff base adducts of amines with aldehydes or ketones which possess improved stability and other desirable properties. The present invention is particularly concerned with economical and efficient methods of producing large quantities of such adduct products on a commercial scale. The above-mentioned technical field is concerned in particular with those adducts having an amine component which is a protein of recognized value in the treatment of animals and humans and in which the adduct product has improved properties relating to its administration and pharmacokinetics.

The present invention is based on the unexpected discovery that said above-mentioned adduct products may be produced in a facile, reproducible and transposable manner with quantitative yields by utilizing freeze-drying, spray-drying or related methods to carry out the basic reaction; and maintaining the pH of the reaction mixture at 7.0 or higher; while at the same time requiring that the aldehyde reactant be selected from aromatic ortho-hydroxy aldehydes. This discovery is broadly applicable to all protein reactants that satisfy certain criteria relating to their practicability that are below-described in more detail. The present invention relates to, e.g., the production of a Schiff base adduct of porcine somatotropin and the aromatic ortho-hydroxy aldehyde, o-vanillin. Porcine somatotropin is a growth hormone which is used for improving feed efficiency in swine.

### BACKGROUND OF THE INVENTION

It is known in the art most pertinent to the present invention that an amine compound, especially a protein, may be improved with regard to its stability and handling characteristics by reacting it with an aldehyde or ketone. For example, cytochrome c has been reacted with salicylaldehyde in an easily reversible process which allows study of the effects of charge modification on the properties of the protein.

Unlike most of the descriptions in the technical literature, that in Williams and Jacobs, Biochim. Biophys. Acta, 154 (1968) 323-331, involves isolation of the Schiff base adduct final product. The mixture of salicylaldehyde and cytochrome c is precipitated and complete conversion may be inferred from the long equilibrium times which were used. The adduct formation involved in this disclosure may be illustrated by the following partial formula:

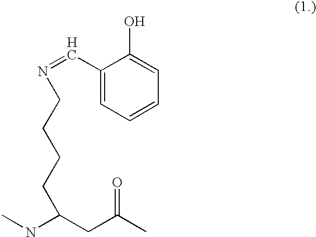

wherein the primary amine is the c-amino group on the lysine molecule which has reacted with the carbonyl moiety of the salicylaldehyde molecule to form an imine, which may be represented as R—(R—)C═N—R. Such imines are usually referred to as Schiff bases and their preparation generally takes place with acid or base catalysis, or with heat. Formation of the Schiff bases is typically driven to completion by precipitation of the imine, removal of water, or both.

As another example of such utilization in the art, sickle erythrocytes have been treated with a variety of aldehydes and ketones to form imine linkages with the amino groups of intracellular hemoglobin. See Zaugg et al., J. Biol. Chem., 252(23) (1977) 8542-8548. Aromatic aldehydes were found to be more reactive than their aliphatic counterparts and ketones were found to be unreactive. The impact of ring substitution on such reactivity conformed to normal expectations regarding electronic and steric effects. In particular, 2,4-dihydroxybenzaldehyde and o-vanillin markedly increased the oxygen affinity of hemoglobins A and S. However, there was no suggestion that o-hydroxy aldehydes would be essential to obtaining quantitative yields in manufacturing Schiff base adducts with proteins.

There are occasional examples in the technical literature of Schiff base adducts with amines other than proteins, e.g., small molecule pharmaceuticals. Fujiwara et al in Chem. Pharm. Bull., 30 (1982) 3310, and in Chem. Pharm. Bull. 31(4) (1983) 1335-1344, refer to the formation of adducts of cephalexin, an antibiotic cephalosporin, and aldehydes. However, there is no suggestion of using o-hydroxy aldehydes; and while the products are obtained by freeze-drying of alkaline solutions thereof; this reference does not suggest the preparation process of the present invention and the quantitative yields obtained thereby.

Schiff bases have been utilized heretofore in analytical procedures for the determination of protein molecular masses as welt as the measurement of the number of primary amino sites (N-terminus plus lysine residues) in a protein. See, e.g., Le Blanc et al. in Anal. Chem., 66 (1994) 3289-3296, which concerns the electrospray mass spectrometric study of protein-ketone equlibria in solution. Acetone is used but aromatic o-hydroxy aldehydes are not suggested.

Electrospray mass spectrometric analysis is used to examine large proteins, e.g., insulin, ubiquitin and hemoglobin, and is also used in conjunction with the process of the present invention in order to provide an accurate and precise means of determining the extent to which Schiff base adducts have been formed. Traditional methods for determining the amount of Schiff base formation between aldehydes and amines are not effective when the amine is a large protein, since these techniques are typically solution methods, and when an isolated Schiff base adduct is dissolved in water, the reverse reaction takes place resulting in an equilibrium mixture. However, Le Blanc uses acetone and does not suggest aromatic o-hydroxy aldehydes.

Schiff base-linked conjugates have been used as a linker between a targeting protein and one or more diagnostic or therapeutic agents. See, e.g., Reed, U.S. Pat. No. 5,633,351. The targeting protein binds to a defined population of cells, such as a receptor or enzyme substrate, and the therapeutic agent is a drug, toxin or radionuclide, while the diagnostic agent is a radionuclide. The Schiff base linkage involved has the following structure:

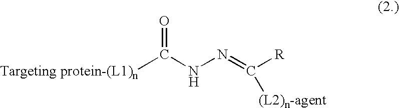

wherein “L1” and “L2” are heterobifunctional linkers having a hydrazide or aldehyde/ketone active group at one end of the linker. However, there is no suggestion of the use of an aromatic o-hydroxy aldehyde at a pH ≧7.0 in order to obtain quantitative yields of a Schiff base adduct final product.

A stabilized somatotropin for parenteral administration is referred to in Clark et al., U.S. Pat. No. 5,198,422, wherein the preferred aromatic aldehyde is said to be 2-hydroxy-3-methoxy benzaldehyde, i.e., o-vanillin. However, Clark et al., refer only to the therapeutic advantages of the somatotropin growth hormone obtained when the product is isolated in crystalline form. While isolation using lyophilization is mentioned in general, it is clear that the methods of isolation contemplated by Clark et al., are of the “desiccation” type, i.e., involving drying over long periods, which is exemplified by drying overnight in a vacuum oven. This reference, consequently, teaches away from the preparation process of the present invention.

There are only limited references in the technical literature to Schiff base adducts prepared by spray-drying. See, e.g., Tomlinson et al., Food Chemistry, 48 (1993) 373-379, which refers to spray-drying an aqueous solution of glucose and glycine. This process produces a brown powder potentially useful in food coloration. The actual chemical process involved is the Maillard or “browning” reaction in which amino groups of proteins react with hydroxyl groups of sugars, forming brown pigments.

The above-mentioned Tomlinson et al. refers to and is based upon the earlier work of Baines et al., U.S. Pat. No. 4,886,659 which is also concerned with the production of colored compounds for use in food chemistry. Baines et al. suggest that colors can be produced from Maillard starting materials under the very short-lived reaction conditions of spray-drying, e.g., a reaction time of less than ten seconds or sometimes less than one second before all of the water is evaporated, effectively ending the reaction.

Use of a stationary spray nozzle or a spinning disc is also referred to, with setting adjustments to control droplet size, dry particle size and other droplet characteristics. The reaction temperature is believed to approximate the outlet air temperature. Preheating of the aqueous solution is also mentioned, e.g., up to 60° C. before feeding to the spray drier, and product moisture content is said to be 3.5-15% by weight. A spinning disc spray drier is also referred to, with a disc speed of 35,000-40,000 rpm.

However, Tomlinson et al. and Baines et al. do not suggest the preparation process of the present invention because they are concerned with the Maillard reaction, in all respects a totally different process. The Maillard reaction is typically irreversible and leads to formation of oligomers. These characteristics limit the usefulness of the Maillard reaction to a preparative procedure for dark pigments.

Dhont, Proc. Int. Symp. Aroma Research, Zeist, (1975) 193-194, refers to work on the aromatization of synthetic foods such as those obtained from soya bean protein. The freeze-drying of a solution of albumin and vanillin is mentioned, with about 90% of the vanillin added being bound by the protein, although it is noted that the protein retains some of the vanillin by encapsulation or adsorption. Formation of Schiff bases is proposed; however, vanillin is not an o-hydroxy aldehyde and complete conversion of the reactants to Schiff bases is not obtained. Accordingly, the process utilized by Dhont is not the same as, nor does it suggest that of the present invention.

### SUMMARY OF THE INVENTION

The present invention relates to a novel improved process for preparing Schiff base condensation adduct final products whose components comprise a protein having beneficial activity in animals, and an aromatic o-hydroxy aldehyde. The process of preparation of the present invention provides substantially quantitative formation of condensation adduct and improved overall yields of final product.

The process of the present invention further relates a method of manufacturing condensation adduct final products which is facile, reproducible, transposable, efficient and economical. Said process comprises bringing together the above-mentioned components in an aqueous environment at a pH of 7.0 or higher to form a reaction mixture. The solvent for the reaction mixture is water, i.e., the medium in which the reaction takes place, including the water of condensation formed during the reaction, said reaction taking place under conditions effective to drive said condensation reaction substantially to completion by removing from about 97.0% to about 99.9% by weight, preferably from about 98.0% to about 99.0% by weight of the water present during said condensation reaction, consistent with maintaining the integrity of the condensation reactants and adduct final product, with resulting yield of said condensation adduct final product of equal to or greater than about 98.5% by weight, preferably equal to or greater than about 99.5% by weight based on the weight of the reactants.

The above-described condensation reaction may also be carried out under conditions of reduced moisture whereby the rate of water removal is accelerated and the overall amount removed is increased. It is provided that, consistent with the goal of driving the condensation reaction to completion by eliminating from about 97.0% to about 99.9% by weight of the water present, that the amount of moisture present in the condensation adduct final product will correspondingly be from 3.0% to 0.001% by weight based on the weight of the final product, preferably from 2.0% to 3.0% by weight, based on the weight of said final product. After the condensation reaction is complete the amount of moisture present may be lowered to from 0.1% to 0.001% by weight, or from 0.05% to 0.005% by weight, or even as low as from 0.03% to 0.01% by weight, based on the weight of the final product. Further, substantially higher amounts of moisture may also be present where required for protein stability, in the range of from 3.0% to 20.0% by weight, preferably from 5.0% to 15.0% by weight, and more preferably from 8.0% to 12.0% by weight, based on the weight of the final product.

Aromatic o-hydroxy aldehydes useful in the above-described condensation process preferably comprise one or more compounds of the formula:

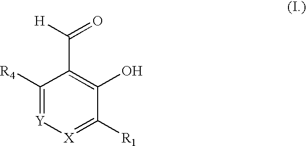

wherein:

R₁ and R₄ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂-C₆)alkenyl; —C(═O)OR₇; —OC(═O)R₇; —S(═O)₂; —S(═O)₂N(R₇)(R₉); —S(═O)₂R₇; —S(═O)₂OR₇; —C(═O)NR₇R₉; —C(═O)R₉; and —N(R₇)(R₉), where R₇ is hydrogen or (C₁-C₄)alkyl and R₉ is (C₁-C₄) alkyl; wherein: said alkyl, cycloalkyl and alkenyl groups defining R₁ and R₄ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl; and

X and Y are independently N, or CHR₂ or CHR₃, respectively, where R₂ and R₃ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂-C₆)alkenyl; —C(═O)OR₁₁; —OC(═O)R₁₁; —S(═O)₂; —S(═O)₂N(R₁₁)(R₁₃); and —N(R₁₁)(R₁₃), where R₁₁ is hydrogen or (C₁-C₄)alkyl and R₁₃ is (C₁-C₄)alkyl; and wherein said alkyl, cycloalkyl and alkenyl groups defining R₂ and R₃ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl;

Preferably, R₁ and R₄ are independently hydrogen; hydroxy; trifluoromethyl; (C₁-C₄)alkyl; (C₁-C₄)alkoxy; —C(═O)OR₇; or —N(R₇)(R₉), where R₇ is hydrogen or (C₁-C₂)alkyl and R₉ is (C₁-C₂); and more preferably R₁ and R₄ are independently hydrogen; hydroxy; (C₁-C₂)alkyl; (C₁-C₂)alkoxy; carboxyl or methylamino, in which case R₇ is hydrogen and R₉ is methyl. Preferably, when R₁ and R₄ are defined as alkyl and are substituted, there is a single substituent selected from hydroxy; (C₁-C₂)alkoxy; carboxyl; amino disubstituted by (C₁-C₂)alkyl; and sulfonamido disubstituted by (C₁-C₂)alkyl; and more preferably said single substituent is selected from hydroxy, methoxy, and dimethylamino.

Preferably, one of X or Y is N and the other is CHR₂, or CHR₃, respectively; more preferably X is CHR₂ and Y is CHR₃, wherein R₂ and R₃ are preferably independently hydrogen; hydroxy; halo; trifluoromethyl; (C₁-C₄)alkyl; (C₁-C₄)alkoxy; —C(═O)OR₁₁; —S(═O)₂N(R₁₁)(R₁₃); or —N(R₁₁)(R₁₃), where R₁₁ is preferably hydrogen or (C₁-C₂)alkyl and R₁₃ is (C₁-C₂)alkyl; more preferably still R₂ and R₃ are independently hydrogen; hydroxy; C₁-C₂)alkyl; (C₁-C₂)alkoxy; carboxyl; or methylamino, in which case R₁₁ is hydrogen and R₁₃ is methyl.

Preferably, when R₂ and R₃ are defined as alkyl and are substituted, there is a single substituent selected from hydroxy; (C₁-C₂)alkoxy; carboxyl; amino disubstituted by (C₁-C₂)alkyl; and sulfonamido disubstituted by (C₁-C₂)alkyl.

Most preferably, said o-hydroxy aldehydes comprise o-vanillin; salicylaldehyde; 2,3-dihydroxybenzaldehyde; 2,6-dihydroxybenzaldehyde; 2-hydroxy-3-ethoxybenzaldehyde; or pyridoxal; which may be represented by the following structural formulas:

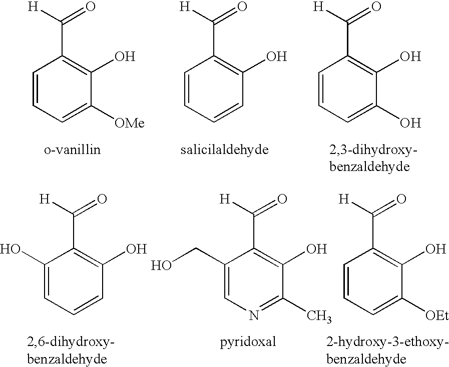

Further, the protein component of the Schiff base condensation adduct final product comprises a peptide having beneficial activity in animals, including utility as a growth promotant in animals employed for the production of food, as well as therapeutic utility as a veterinary product for the treatment and prevention of numerous diseases and adverse conditions. The protein components also have utility as therapeutic agents in the treatment and prevention of diseases and adverse conditions in humans.

The protein components are primary amines in chemical structure and may have as few as two amino acids up to several hundred to as many as a thousand or more amino acids. Said protein components and the condensation adduct final products which they form as provided herein, are of recognized value in the treatment of animals and humans.

The following particular proteins are especially suitable for use in the present invention:

proteinaceous endogenous and synthetic opioid analgesics and antagonists comprising enkephalins, endorphins, and dynorphins which are selective and nonselective agonists and antagonists of the μ, κ, and δ opioid receptor subtypes, including [Leu⁵] and [Met⁵]enkephalin; dynorphin A and B; α- and β-neoendorphin; [D-Ala², MePhe⁴,-Gly(ol)⁵]enkephalin (DAMGO); [D-Pen², D-Pen⁵]enkephalin (DPDPE); [D-Ser², Leu⁵]enkephalin-Thr⁶ (DSLET); [D-Ala², D-Leu⁵]enkephalin (DADL); D-Phe-Cys-Tyr-D-Trp-Orn-Thr-Pen-Thr-NH₂ (CTOP); [D-Ala², N-MePhe⁴, Met(O)⁵-ol]enkephalin (FK-33824); Tyr-D-Ala-Phe-Asp-Val-Val-Gly-NH₂ ([D-Ala²]deltorphin I; Tyr-D-Ala-Phe-Glu-Val-Val-Gly-NH₂ ([D-Ala², Glu⁴]deltorphin II; Tyr-Pro-Phe-Pro-NH₂ (morphiceptin); Tyr-Pro-MePhe-D-Pro-NH₂ (PL-017); and [D-Ala², Leu⁵, Cys⁶]enkephalin;

autocoids including bradykinin and kallidin produced by proteolytic reactions in response to inflammatory events selected from tissue damage, viral infections, and allergic reactions, wherein said proteins act locally to produce pain, vasodilatation, increased vascular permeability and the synthesis of prostaglandins, wherein said proteins have agonist and antagonist activity and are useful for the treatment of male infertility, for the delivery of cancer chemotherapeutic agents beyond the blood-brain barrier, and for the treatment of pain, asthma, and other chronic inflammatory diseases, including: Arg-Pro-Pro-Gly-Phe-Ser-Pro-Phe-Arg (bradykinin); Lys-Arg-Pro-Pro-Gly-Phe-Ser-Pro-Phe-Arg (kallidin); Arg-Pro-Pro-Gly-Phe-Ser-Pro-Phe (des-Arg⁹-bradykinin); Lys-Arg-Pro-Pro-Gly-Phe-Ser-Pro-Phe (des-Arg¹⁰-kallidin); Arg-Pro-Pro-Gly-Phe-Ser-Pro-Leu (des-Arg⁹-[Leu⁸]-bradykinin); Arg-Pro-Pro-Gly-Phe-Ser-[D-Phe]-Phe-Arg ([D-Phe⁷]-bradykinin); and [D-Arg]-Arg-Pro-Hyp-Gly-Thi-Ser-Tic-Oic-Arg (HOE 140), where Hyp is trans-4-hydroxy-Pro; Thi is β-(2-thienyl)-Ala; Tic is [D]-1,2,3,4-tetrahydroquinolin-3-yl-carbonyl; and Oic is (3 as,7 as)-octahydroindol-2-yl-carbonyl;

proteins active at vasopressin receptor subtypes V₁ and V₂ which mediate pressor responses and antidiuretic responses, respectively, including V₁ antagonists beneficial in the treatment of congestive heart failure, hypertension, and postoperative ileus and abdominal distension, V₂ agonists used to treat central diabetes insipidus by controlling polyuria and polydipsia, and to treat bleeding disorders including von Willebrand's disease, including the specific naturally-occurring vasopressin-like peptides: arginine vasopressin (AVP) of the following formula:

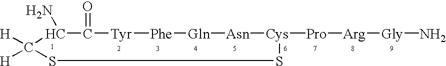

and lypressin ([Lys⁸]-AVP; synthetic vasopressin peptides: V₁ₐ-selective agonist [Phe², Ile², Orn⁸]AVP; V_{1b}-selective agonist deamino [D-3-(3′-pyridyl)-Ala²]AVP, V₂-selective agonists desmopressin (dDAVP), and deamino[Val⁴, D-Arg⁸]AVP; and peptide antagonists including V₁ₐ-selective antagonist d(CH₂)₅[Tyr(Me)²]AVP of the formula:

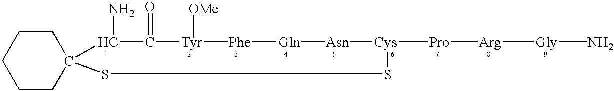

and V_{1b}-selective antagonist dp[Tyr(Me)²]AVP; and V₂-selective antagonists des Gly-NH₂⁹-d(CH₂)₅[D-Ile, Ile⁴]AVP, and d(CH₂)₅[D-Ile², Ile⁴, Ala-NH₂⁹]AVP;

pentagastrin used as an indicator of gastric secretion of the formula: N-t-butyloxycarbonyl-β-Ala-Trp-Met-Asp-Phe-NH₂;

octreotide useful in treating the symptoms of tumors of the gastrointestinal tract, diarrhea refractory to other treatment, motility disorders, and gastrointestinal bleeding of the formula: L-cysteinamide-D-Phe-L-Cys-L-Phe-D-Trp-L-Lys-L-Thr-N-[2-hydroxy-1-(hydroxymethyl)propyl]-. cyclic (2→7)-disulfide, [R—(R*,R*)]—;

antibody reagents useful as immunosuppressive agents including antithymocyte globulin; muromonab-CD3 monoclonal antibody; and Rh₀(D) immune globulin; and protein immunostimulants useful in treating immunodeficiency states, including immune globulin;

cytokines produced by leukocytes and having a variety of immunoregulatory effects, including: interferons, colony-stimulating factors, and interleukins, and specifically α-interferon; interferon-γ (IFN-γ); granulocyte colony-stimulating factor (G-CSF); granulocyte macrophage colony-stimulating factor (GM-CSF); and interleukin-1 (IL-1) through interleukin-12 (IL-12);

hematopoietic growth factors involved in the regulation of the process whereby mature blood cells are continuously replaced, useful in the treatment of primary hematological diseases and uses as adjunctive agents in the treatment of severe infections and in the management of patients who are undergoing chemotherapy or marrow transplantation, including specifically: growth factors including erythropoietin (EPO); stem cell factor (SCF); interleukins (IL-1-12); monocyte/macrophage colony-stimulating factor (M-CSF, CSF-1); P1XY321 (GM-CSF/IL-3 fusion protein); and thrombopoietin;

thrombolytic proteins useful for dissolving both pathological thrombi and fibrin deposits at sites of vascular injury, including streptokinase; tissue plasminogen activator (t-PA); and urokinase;

anterior pituitary hormones and the hypothalamic factors that regulate their use comprising: (a) somatotropic hormones including growth hormone (GH), prolactin (Prl), and placental lactogen (PL); (b) glycoprotein hormones including luteinizing hormone (LH), follicle-stimulating hormone (FSH), and thyroid-stimulating hormone (TSH); and (c) POMC-derived hormones including corticotropin (ACTH), α-melanocyte-stimulating hormone (α-MSH), β-melanocyte-stimulating hormone (β-MSH), β-lipotropin (β-LPH), and γ-lipotropin (γ-LPH); the hypothalamic factors regulating release of said hormones, including growth hormone-releasing hormone (GHRH), luteininzing hormone releasing hormone (LHRH), insulin-like growth factor (IGF-1 and IGF-2), somatostatin, and gonadotropin-releasing hormone (GnRH);

growth hormone useful as replacement therapy in growth-hormone deficient children, including: somatostatin, the synthetic analogue of somatostatin, octreotide; gonadotropic hormones including LH, FSH, and corionic gonadotropin (GC) useful in the diagnoses of reproductive disorders and in the treatment of infertility, including: urofollitropin, a human menopausal gonadotropin (hMG) from which substantially most of the LH has been removed useful for inducing ovulation, and gonadorelin, a synthetic human GnRH useful for stimulating gonadotropin secretion; synthetic GnRH agonists including: leuprolide, histrelin, nafarelin, and goserelin useful in treating endocrine disorders that are responsive to reductions in gonadal steroids;

thyrotropin (TSH), the secretion of which is controlled by thyrotropin-releasing hormone (TRH), useful for hormone replacement therapy in patients with hypothyroidism and for TSH suppression therapy in patients with nontoxic goiter or after treatment for thyroid cancer;

insulin for treating insulin-dependent diabetes mellitus patients and non-insulin-dependent diabetes mellitus patients; glucagon which has a physiological role in the regulation of glucose and ketone body metabolism, useful in treating severe hypoglycemia, and by radiologists for inhibiting the gastrointestinal tract; somatostatin, useful for blocking hormone release in endocrine-secreting tumors, including insulinomas, glucagonomas, VIPomas, carcinoid tumors, and somatotropinomas, and the synthetic analogue, octreotide;

calcitonin, a hormone acting specifically on osteoclasts to inhibit bone resorption, is useful in managing hypercalcemia and in disorders of increased skeletal remodeling, including Paget's disease; parathyroid hormone, useful in the treatment of patients with spinal osteoporosis;

aldesleukin, 125-L-serine-2-133-interleukin 2, useful as an antineoplastic agent and as an immunostimulant; alglucerase, a monomeric glycoprotein of 497 amino acids and a modified form of human placental tissue β-glucocerebrosidase, is useful as a replenisher of the glucocerebrosidase enzyme; alsactide, a synthetic corticotropin analogue: 1-β-Ala-17[L-2,6-diamino-N-(4-aminobutyl)hexanamide]-α¹⁻¹⁷-corticotropin; alteplase, a serine protease of 527 amino acids whose sequence is identical to the naturally occurring protease produced by endothelial cells in vessel walls, useful as a plasminogen activator; alvircept sudotox, a synthetic chimeric protein engineered to link the first 178 amino acids of the extracellular domain of CD₄ via two linker residues to amino acids 1-3 and 253-613 of Pseudomonas exotoxin A, useful as an antiviral agent; amlintide, a protein of 37 amino acids, useful as an antidiabetic agent; amogastrin: N-carboxy-L-Trp-L-Met-L-α-Asp-3-phenyl-L-Alaninamide; anakinra: N²-L-Met-interleukin 1 receptor antagonist useful as a nonsteroidal anti-inflammatory and as a suppressant for treating inflammatory bowel disease; anaratide acetate, atriopeptin-21 (rat), N-L-Arg-8-L-Met-21a-L-Phe-21b-L-Arg-21c-L-Tyr-, acetate, useful as an antihypertensive agent and as a diuretic; angiotensin amide, angiotensin II, 1-L-Asn-5-L-Val-, useful as a vasoconstrictor; aprotinin, a pancreatic trypsin inhibitor having 58 amino acids, useful as an enzyme inhibitor (proteinase); arfalasin, 1-succinamic acid-5-L-Val-8-(L-2-phenylglycine)angiotensin II, useful as an antihypertensive agent; argipressin tannate, vasopressin, 8-L-Arg-, tannate, useful as an antidiuretic; aspartocin, oxytocin, 4-L-Asn-, is useful as an antibiotic agent produced by Streptomyces griseus; atosiban, oxytocin, 1-(3-mercaptopropanoic acid)-2-(O-ethyl-D-Tyr)-4-L-Thr-8-L-Orn-, useful as an oxytocin antagonist; avoparcin, a glycopeptide antibiotic obtained from Streptomyces candidus; basifungin, N-[(2R,3R)-2-hydroxy-3-MeVal]-N-L-MeVal-L-Phe-N-L-MePhe-L-Pro-L-allo-Ile-N-L-MeVal-L-Leu-3-hydroxy-N-L-MeVal α₁-lactone, useful as an antifungal agent; becaplermin, recombinant human platelet-derived growth factor B, a recombinant protein produced by genetically engineered Saccharomyces cerevisiae similar in amino acid composition and biological activity to endogenous human PDGF-BB homodimer, useful for treating chronic dermal ulcers by promoting proliferation of mesenchymally-derived cells; bivalirudin, an anticoagulant, antithrombotic agent having 20 amino acids; carbetocin, 1-butyric acid-2-[3-(p-methoxyphenyl)-L-Ala]oxytocin; cargutocin, 1-butyric acid6-(L-2-aminobutyric acid)-7-glycineoxytocin; ceruletide, 5-O-L-Pro-L-Gln-L-α-Asp-L-O-sulfo-L-Tyr-L-Thr-L-Gly-L-Trp-L-Met-L-α-Asp-L-Phe-amide, useful as a gastric secretory stimulant; cetermin, transforming human growth factor β2 having 112 amino acids; cilmostim, 1-233-colony-stimulating factor 1 (human clone p3ACSF-69 protein moiety), cyclic (7→90), (48→139), (102→146)-tris(disulfide) dimer, useful as a hematopoietic agent (macrophage colony-stimulating factor); colistimethate sodium, a colistin A component useful as an antibacterial agent; corticorelin, ovine triflutate, corticotropin-releasing factor (sheep), trifluoroacetate salt, useful as a diagnostic aid for adrenocortical insufficiency and Cushing's syndrome, and as a corticotropin-releasing hormone; cosyntropin, tetracosactide acetate, α¹⁻²⁴-corticotropin, useful as an adrenocorticotropic hormone; cyclosporin, a cyclic protein containing 11 amino acids and a 3-hydroxy-4-methyl-2-(methylamino)-6-octenoyl moiety at the 6-position, useful as an immunosuppressant; dacliximab (Ro-24-7375), a humanized anti-TAC monoclonal antibody comprised of four subunits linked via disulfide bridges and a molecular weight of approximately 150 kD, useful as an immunosuppressant; daclizumab; daptomycin, a proteinaceous antibacterial agent; desirudin, 63-desulfohirudin from Hirudo medicinalis comprising 63 amino acids, useful as an anticoagulant; deslorelin, luteinizing hormone-releasing factor (pig) comprising 9 amino acids, useful as an LHRH agonist; desmopressin acetate, vasopressin, 1-(3-mercaptopropanoic acid)-8-D-Arg-, monoacetate salt, trihydrate, comprising 9 amino acids, useful as an antidiuretic; detirelix acetate comprising 10 amino acids, useful as an LHRH antagonist; dumorelin, 27-L-Leu-44a-Gly growth hormone-releasing factor (human); elcatonin, 1-butyric acid-7-(L-2-aminobutyric acid)-26-L-Asp-27-L-Val-29-L-Ala calcitonin (salmon); emoctakin, interleukin 8 (human) comprising 72 amino acids with two Cys bridges; epoetin alfa, a 165 amino acid glycoprotein that regulates red blood cell production and is produced by Chinese hamster ovary cells into which the human erythropoietin gene has been inserted, useful as an anti-anemic and hematinic agent; ersofermin, recombinant human basic fibroblast growth factor (bFGF) comprising 157 amino acids, a non-glycosylated protein isolated from human placenta and cloned and expressed in E coli, useful as a wound healing agent; felypressin is vasopressin, 2-L-Phe-8-L-Lys comprising 9 amino acids, useful as a vasoconstrictor; filgrastim, a single chain 175 amino acid polypeptide, non-glycosylated and expressed by E. coli, useful as an antineutropenic agent and as a haematopoietic stimulant; glucagon, a single chain protein of 29 amino acids, useful an antidiabetic agent; gonadorelin acetate, the diacetate salt of luteininzing hormone-releasing factor acetate comprising 10 amino acids, useful as a gonad-stimulating principle; goserelin, luteinizing hormone-releasing factor (pig) comprising 9 amino acids, useful as an LHRH agonist; histrelin, luteinizing hormone-releasing factor (pig) comprising 9 amino acids, useful as an LHRH agonist; imiglucerase, 495-L-Histidineglucosylceramidase placenta isoenzyme protein, useful as an enzyme replenisher for glucocerebrosidase; insulin, dalanated, an insulin derivative prepared by removal of the C-terminal alanine from the B chain of insulin, useful as an antidiabetic agent; interferon alfa-2a, interferon αA (human leukocyte protein moiety reduced) comprising 165 amino acids, useful as an antineoplastic agent and as a biological response modifier; interferon alfa-2b, interferon α2b (human leukocyte clone Hif-SN206 protein moiety reduced) comprising 165 amino acids, also useful as an antineoplastic agent and as a biological response modifier; interferon beta-1 a, a glycosylated polypeptide consisting of 166 amino acid residues produced from cultured Chinese hamster ovary cells containing the engineered gene for human interferon beta, also useful as an antineoplastic agent and as a biological response modifier; interferon beta-1b, a non-glycosylated polypeptide consisting of 165 amino acid residues produced from E. coli, also useful as an immunomodulator; interferon gamma-1b, 1-139 interferon γ (human lymphocyte protein moiety reduced), N²-L-Met, useful as an antineoplastic agent and as an immunomodulator; iroplact, N-methionylblood platelet factor 4 (human subunit) comprising 71 amino acid residues having two Cys bridges; lanoteplase, a tissue plasminogen activator protein derived from human t-PA by deletion of the fibronectin-like and the EGF-like domains and mutation of Asn 117 to Gln 117, produced by expression in a mammalian host cell of a DNA sequence encoding the peptide sequence, useful as a plasminogen activator and thrombolytic agent; lanreotide acetate comprising 8 amino acids and one disulfide bridge, useful as an antineoplastic agent; lenograstim, a glycoprotein consisting of 174 amino acid residues produced in Chinese hamster ovary cells by expression of a human granulocyte colony-stimulating factor-cDNA derived from a human oral cavity squamous cell line-mRNA, useful as an antineutropenic agent and as an haematopoietic stimulant; lutrelin acetate, a luteinizing hormone-releasing factor (pig) comprising 9 amino acids, useful as an LHRH agonist; molgramostim, a colony-stimulating factor 2 (human clone pHG₂₅ protein moiety reduced) comprising 127 amino acids, useful as an antineutropenic agent and as an haematopoietic stimulant; murodermin, an epidermal growth factor (mouse salivary gland); nafarelin acetatem, luteinizing hormone-releasing factor (pig) comprising 9 amino acids, useful as an LHRH agonist; nagrestipen, 26-L-Alaninelymphokine MiP 1α (human clone pAT 464 macrophage inflammatory comprising 69 amino acids and having two disulfide bridges; pepstatin, N-(3-methyl-1-oxobutyl)-L-Val-L-Val4-amino-3-hydroxy-6-methylheptanoyl-L-Ala-4-amino-3-hydroxy-6-methylheptanoic acid, useful as a pepsin enzyme inhibitor; pramlintide, a protein comprising 37 amino acids and having one disulfide bridge, useful as an antidiabetic agent; proinsulin human, proinsulin (pig) comprising 86 amino acid residues and having three disulfide bridges, useful as an antidiabetic agent; sargramostim, colony-stimulating factor 2 (human clone pHG25 protein moiety), 23-L-Leu-, a single chain, glycosylated polypeptide of 127 amino acid residues expressed from Saccharomyces cerevisiae, useful as an antineutropenic agent and a haematopoietic stimulant; naturally occurring and synthetically, including recombinantly derived human and animal somatotropins (growth hormones), especially bovine and porcine somatotropins; somagrebove, somatotropin (ox reduced), 1-[N²-L-Met-L-α-Asp-L-Glutamine]- comprising 191 amino acids, useful as a galactopoietic agent especially for veterinary use; somalapor, somatotropin (pig clone pPGH-1 reduced), N-L-Alanyl-growth hormone comprising a total of 191 amino acids, useful as a hormone (growth, porcine); somatrem, somatotropin (human), N-L-Met- comprising 191 amino acids having two disulfide bridges, useful as a growth hormone; somatotropin, a single polypeptide chain comprising 191 amino acids having the normal structure of the principal growth stimulating hormone obtained from the anterior lobe of the human pituitary gland, useful as a growth hormone; somatotropin, available in recombinant form; somavubove, somatotropin, (ox), 127-L-Leu-, one of the four naturally occurring molecular variants in bovine pituitary somatotropin, useful as a galactopoietic agent; somenopor, somatotropin (pig clone pPGH-1 reduced), N-L-Ala-32-de-L-Glu-33-de-L-Arg-34-de-L-Ala-35-de-L-Tyr-36-de-L-Ile-37-de-L-Pro-38-de-L-Glu- comprising 190 amino acids, useful as a porcine growth hormone; sometribove, somatotropin (ox), 1-L-Met-127-L-Leu- comprising 191 amino acids, useful as a veterinary growth stimulant; sometripor, somatotropin (pig recombinant) C₉₇₉H₁₅₂₇N₂₆₅O₂₈₇S₈; somfasepor, somatotropin (pig recombinant) C₉₃₈H₁₄₆₅N₂₅₇O₂₇₈S₆; somidobove, somatotropin (ox recombinant) C₁₀₂₀H₁₅₉₆N₂₇₄O₃₀₂S₉; teprotide, bradykinin potentiator B, 2-L-Trp-3-de-L-Leu-4-de-L-Pro-8-L-Glutamine- comprising 9 amino acids, useful as an angiotensin-converting enzyme inhibitor; teriparatide, a protein comprising 34 amino acids, useful as a bone resorption inhibitor and an osteoporosis therapy adjunct; thymalfasin, thymosin al (ox) comprising 28 amino acids, useful as an antineoplastic agent, in treating hepatitis and infectious diseases, and as a vaccine enhancer; thymopentin, a pentapeptide useful as an immunoregulator; triptorelin, luteinizing hormone-releasing factor (pig), 6-D-Trp comprising 10 amino acids, useful as an antineoplastic agent; vapreotide comprises 8 amino acids having one disulfide bridge, useful as an antineoplastic agent; vasopressin in the 8-L-Arg- or 8-L-Lys- form comprising 9 amino acids having one disulfide bridge, useful as an antidiuretic hormone; myoglobin; hemoglobin; β-lactoglobulin; immunoglobulin-G (IgG); antihemophilic factor (Factor VIII); lysozyme; ubiquitin; platelet-activating factor (PAF); tumor necrosis factor-α (TNF-α); tumor necrosis factor-β (TNF-β); macrophage inflammatory protein (MIP); heparin; eosinophil cationic protein (ECP); recombinant factor IX; monoclonal antibody for non-Hodgkin's B-cell lymphoma; interferon alpha, useful for treating hepatitis C; and fibroblast-derived artificial skin for treating wounds and burns.

The conditions which are effective to drive the condensation reaction of the inventive process substantially to completion comprise those which change the water present from the liquid phase to the gaseous or solid phase whereby it is removed from the environment of said condensation reaction. In order to be successful in the process of the present invention, said conditions must also be characterized by scalability, i.e., the ability to be readily and efficiently adapted to large, manufacturing scale production, and by reproducibility, i.e., the ability to be carried out successively without substantial deviation in end result. Accordingly, said conditions are those which optimize the energy input to the process necessary most efficiently to separate the water of the aqueous environment in which the condensation reaction takes place, including water produced by said condensation reaction itself, from the starting material reactants and the condensation adduct final product.

At temperatures above 0° C. the conditions which optimize energy input to the process comprise (a) heating said reaction mixture in said aqueous environment to the highest temperature consistent with maintaining the integrity of the protein starting material reactant and the condensation adduct final product, as well as consistent with optimal efficiencies and economies for carrying out said preparation process including said condensation reaction; (b) subdividing said reaction mixture in said aqueous environment into the smallest droplets consistent with maintaining the integrity of the protein starting material reactant and the condensation adduct final product, as well as consistent with optimal efficiencies and economies for carrying out said preparation process including said condensation reaction; and (c) providing said droplets thus formed with the highest comparative velocity, referenced to a gas inert thereto through which they pass, which is consistent with maintaining the integrity of the protein starting material reactant and the condensation adduct final product, as well as consistent with optimal efficiencies and economies for carrying out said preparation process including said condensation reaction.

Further, the reaction mixture is heated to a temperature of from 25° C. to 125° C., preferably from 40° C. to 120° C., more preferably from 50° C. to 115° C., more preferably still from 60° C. to 110° C., and most preferably from 75° C. to 105° C., while maintaining the aqueous environment in the liquid phase by the application of elevated pressure where necessary. It will usually be more beneficial to utilize lower, i.e., reduced pressures, however, in light of the fact that one of the reactants is a protein. Reduced pressure permits the input of essentially the same amount of energy to the system, while maintaining a lower temperature therein, in order to change the reaction mixture from the liquid to the vapor phase. Thus, it will be understood that caution must be used with regard to any high temperatures which the reaction mixture is permitted to encounter. The highest of the above-mentioned temperatures can, in most cases, be maintained for only a very brief time, usually a matter of seconds. Maintaining lower temperatures in the reaction mixture, optionally assisted by the use of reduced pressure, may prove advantageous or even necessary where reactants or final products have melting points sufficiently low that they pose processing problems.

Still further, the reaction mixture in said aqueous environment is divided into droplets having an average diameter of from 1.0 μm to 5.0 mm, preferably from 10 μm to 1.0 mm, more preferably from 100 μm to 900 μm, more preferably still from 200 μm to 800 μm, and most preferably from 300 μm to 700 μm.

The comparative velocity to which said droplets are subjected is from 0.1 m/sec to 5.0 m/sec, preferably from 0.2 m/sec to 4.0 m/sec, more preferably from 0.3 m/sec to 3.0 m/sec, more preferably still from 0.4 m/sec to 2.0 m/sec, and most preferably from 0.5 m/sec to 1.0 m/sec.

At temperatures of 0° C. and below that said conditions which optimize energy input to the process comprise (a) cooling said reaction mixture in said aqueous environment to a temperature sufficiently low to freeze substantially all of the unbound liquid water present in said aqueous environment, said temperature being consistent with maintaining the integrity of the protein starting material reactant and the condensation adduct final product, as well as consistent with optimal efficiencies and economies for carrying out said preparation process including said condensation reaction; (b) subjecting said thus cooled reaction mixture in said frozen aqueous environment to a reduced pressure in the presence of a gas inert thereto, which is consistent with maintaining the integrity of the protein starting material reactant and the condensation adduct final product, as well as consistent with optimal efficiencies and economies for carrying out said preparation process including said condensation reaction.

Further, the reaction mixture is cooled to a temperature of from −110° C. to 0° C., preferably from −45° C. to −5° C., more preferably from −40° C. to −10° C., more preferably still from −35° C. to −15° C., and most preferably from −30° C. to −20° C., while maintaining the aqueous environment in the solid phase.

The reduced pressure to which said cooled reaction mixture in said aqueous environment is subjected is from 5.0 mmHg absolute to 0.0001 mmHg absolute, preferably from 1.0 mmHg absolute to 0.0005 mmHg absolute, more preferably from 0.5 mmHg absolute to 0.001 mmHg absolute, more preferably still from 0.2 mmHg absolute to 0.005 mmHg absolute, and most preferably from 0.1 mmHg absolute to 0.01 mmHg absolute.

The condensation reaction processes of the present invention may also be carried out under conditions of reduced moisture whereby the rate of water removal is accelerated and the overall amount removed is increased. This is consistent with the goal of driving the condensation reaction to completion by eliminating from about 97.0% to about 99.9% by weight, preferably from about 98.0% to about 99.0% by weight of the water already present or produced during said condensation reaction, to assure a rate of conversion to said condensation adduct final product, i.e., with resulting yield of said condensation adduct final product of equal to or greater than about 98.5% by weight, preferably equal to or greater than about 99.5% by weight based on the weight of the reactants. Consistant with that goal, the amount of moisture present in the condensation adduct final product will correspondingly be from 3.0% to 0.001% by weight based on the weight of the final product, preferably from 2.0% to 3.0% by weight, based on the weight of said final product. It is further provided that after the condensation reaction is complete the amount of moisture present may be lowered to from 0.1% to 0.001% by weight, or from 0 05% to 0 005% by weight, or even as low as from 0.03% to 0.01% by weight, based on the weight of the final product. Substantially higher amounts of moisture may be present where required for protein stability, in the range of from 3.0% to 20.0% by weight, preferably from 5.0% to 15 0% by weight, and more preferably from 8.0% to 12.0% by weight, based on the weight of the final product.

The above-described preparation processes including condensation processes may be carried out under conditions of reduced moisture whereby the rate of water removal is accelerated and the overall amount removed is increased. This procedure is consistent with the goal of driving the condensation reaction to completion by eliminating from about 97.0% to about 99.9% by weight, preferably from about 98.0% to about 99.0% by weight of the water already present or produced during said condensation reaction. However, the amount of moisture present in the condensation adduct final product must be consistent with maintaining the integrity of said final product. Accordingly, the desirable levels of moisture in the adduct final product will be in the range of from 3.0% to 20.0% by weight, preferably from 4.0% to 15.0% by weight, and more preferably from 5.0% to 10.0% by weight, based on the weight of the final product. For example, where the product is ovine somatotropin, the amount of moisture present in the final product will be from 6.0 to 9.0% by weight.

Application of the above-described conditions to the preparation processes of the present invention will be effective to remove from about 97.0% to about 99.9% by weight, preferably from about 98.0% to about 99.0% by weight of the water present during said condensation reaction, consistent with maintaining the integrity of the condensation reactants and adduct final product, with resulting yield of said condensation adduct final product of equal to or greater than about 98.5% by weight, preferably equal to or greater than about 99.5% by weight based on the weight of the reactants.

Further, the starting material reactants can also be brought into intimate contact with each other as an aqueous solution immediately prior to or substantially simultaneously with dispersion of the condensation adduct final product in droplet form. This intimate admixture in the form of an aqueous solution is achieved by mechanical action sufficient to bring said starting material reactants into contact with each other while at the same time not mechanically degrading the protein component of said condensation adduct. There are provided guidelines for choosing a mechanical mixing device which has a gentle action in order to avoid significant levels of shear stress in solution. For example, the artisan may choose stationary mixing vessels with rods, paddles or other types of stirrers; continuous mixing apparatus in the form of a trough with agitation means comprising a slow moving worm or baffles which operate in conjunction with rocking of the entire trough; a double-pipe arrangement with the reaction mixture carried in the central pipe and the countercurrent flow heating medium in the annulus between the pipes, with agitation by a shaft rotating in the central pipe which carries blades; a stirred reaction vessel with calanders employed for heating in which the downcomer houses an impeller, with forced circulation increasing the heat transfer to the reaction mixture; mixing devices which concentrate the reaction mixture; and a vacuum reactor vessel with an agitated reactor chamber maintained at low pressure.

The above-mentioned intimate admixture in the form of an aqueous solution is also achieved by inversion of an inverse emulsion in which said starting material reactants have been separated from each other as solutes in the continuous, i.e., solvent phase and in the dispersed, i.e., aqueous phase of said inverse emulsion. Inversion of said inverse emulsion is achieved by rapid distribution of said inverse emulsion into an aqueous system, which as noted is the same as the dispersed phase. The resulting condensation adduct final product derived by any of the above-described methods of admixture is then ready to be dispersed in droplet form under ambient conditions, as further below-described.

The starting material reactants may also be brought into intimate contact with each other in droplet form, i.e., formation of said condensation adduct final product occurs immediately prior to or substantially simultaneously with dispersion of said final product in droplet form. Intimate admixture of said starting material reactants in droplet form is achieved by mechanical action in the form of separate sprays of each said reactant starting material directed in such manner with respect to each other that maximum commingling, collision, and contact of said droplets is achieved. Spraying apparatus for use in this process may comprise mechanical or hydraulic pumping means sufficient to impart the energy necessary to divide an aqueous stream containing said starting material reactants into droplets of the size required to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants. Said pumping means may be used in conjunction with a nozzle means whereby mechanical shearing forces are applied to said aqueous stream of said starting material reactants as a result of which said stream is divided into successively smaller droplet total volumes until the desired droplet size is achieved.

Spraying apparatus may also be used comprising gas stream generators and means for dispersing said aqueous stream of said starting material reactants therein so as to be entrained thereby in droplet form having the desired droplet size. Said gas is substantially inert with respect to said starting material reactants and said condensation adduct final product, and comprises air, nitrogen, or helium, among others, which has been compressed to a pressure sufficiently high to provide a gas stream having the volume and velocity required to entrain said droplets of said starting material reactants and assure a maximum commingling, contact and collision thereof sufficient to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

The spraying apparatus used in the method of the present invention comprises any suitable combination of the above-described gas stream generators and associated dispersing means together with said above-described hydraulic pumping means and associated nozzle means.

The intimate admixture of said starting material reactants in droplet form in accordance with the present invention may also be achieved by mechanical action in the form of a rapidly rotating disc over the surface of which an aqueous stream comprising each said reactant starting material is directed. A separate disc for each reactant starting material may be utilized, or else a single disc is used which is fashioned to accommodate both said reactant starting material aqueous streams. Each said aqueous stream traverses said disc in such manner that it is propelled from the edge of said disc in droplet form. The speed of said rotating disc is varied so as to impart sufficient energy to divide each said aqueous stream into droplets of such size and speed that maximum commingling, collision, and contact of said droplets is achieved. Commingling of said starting material reactants takes place under substantially ambient conditions adjusted with regard to temperature, humidity and pressure so as eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

It is contemplated to be within the scope of the present invention to carry out the above-described preparation processes under substantially ambient conditions. However, in the preferred embodiments of the present invention said substantially ambient conditions are significantly modified so as to improve the rate and total extent of water removal from said condensation reaction and the resultant adduct final product. In particular, said modifications include heating of said reactant starting material aqueous streams and the apparatus means by which they are processed during some or all of the procedures of said preparation methods provided herein. Accordingly, the rates of reaction and extent of conversions to condensation adduct final product are substantially increased.

The preparation process may be modified by applying electrical fields to various parts of the apparatus or materials involved in said preparation process whereby commingling, collision, and contact of said reactant starting material droplets involved is maximized and the yield of said condensation adduct final product is substantially improved.

Apparatus means and process steps may be placed under substantially reduced pressure conditions, particularly those means and steps involved in the reaction of said starting material reactants, in order to achieve maximum commingling, contact and collision thereof sufficient to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

Fluidized bed means may be utilized to improve the rate and extent of water elimination from the droplet condensation adduct final product as well as to improve the yield of said droplet condensation adduct final product to equal to or over about 98.5% by weight based on the weight of said starting material reactants.

The present invention further relates to novel compositions of matter produced by the above-described preparation processes of the present invention comprising Schiff base condensation adducts whose components comprise a protein and an aromatic o-hydroxy aldehyde, wherein said components have formed a reaction mixture and resulting condensation adduct final product under conditions effective to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants. Also provided are said compositions of matter in droplet form having mean diameters in the range of from about 0.1 μm to about 10.0 μm, preferably from about 1 0 to about 5.0 μm, more preferably from about 2.0 μm to about 4.0 μm, and most preferably from about 2.5 μm to about 3.5 μm.

Novel compositions of matter of the present invention include those wherein said protein component thereof can be administered to a animal and thereafter be taken up, beneficially utilized, metabolized and cleared, i.e., eliminated from said animal. Said protein component may have such characteristics before it is reacted with an aromatic o-hydroxy aldehyde to form the improved Schiff base condensation adducts of the present invention; nevertheless, the formation of such a condensation adduct will significantly enhance such properties and characteristics and may thereby render suitable a protein candidate that would otherwise fail to be suitable.

Said protein component has the ability to achieve a beneficial utility in the particular animal or animals to which it is administered, which is most commonly one which is therapeutic. Included are roteins having other biological activities which are of benefit to an animal or to the use of an animal, including protein hormones such as somatotropin which is used to regulate the growth of animals usually kept as domestic stock for food production, and somatotropin administered to such animals has the beneficial utility of increasing feed utilization efficiency and reducing the time necessary to bring such a stock animal to market. Also included are proteins improved by use of the processes of the present invention with respect to both their long term storage stability as well as with respect to enhanced opportunities for their administration to animals by such modes as parenteral solid implants. Still further included are proteins which have recognized utility as therapeutic agents for animals and man, and which may be used with the processes of the present invention.

### DETAILED DESCRIPTION OF THE INVENTION

The present invention is concerned with a process for making Schiff base condensation adducts comprising an o-hydroxy aldehyde and a protein, which represents a significant improvement over processes of preparation known heretofore in the art for making adduct products of this type. Not only is the method of preparation of the present invention more facile in terms of its reproducibility, efficiency, high yields, and transposability, i.e., suitability for scaled up implementation, but the condensation adduct final product of this process also represents a significant and surprising improvement over the products produced by processes of preparation employed in the past. The condensation adduct final product of the present invention results directly from the improved condensation process of the present invention itself with its surprisingly better reproducibility, efficiency, high yields, and transposability, i.e., suitability for scaled up implementation.

As indicated, the present invention involves a significant improvement over Schiff base condensation processes described in the technical literature. Representative of such processes is that referred to in Clark et al. U.S. Pat. No. 5,198,422, in which a stabilized complex comprising a growth hormone, especially porcine somatotropin, pST, and an aromatic aldehyde is prepared, and the final product complex is isolated from aqueous solution as a crystalline product alleged to provide prolonged release of said growth hormone.

The particular procedure by which the final product complex is isolated from aqueous solution in the method of Clark et al. involves removal of the aqueous solvent by evaporation over a substantial period of time, after which the product is recovered by scraping it from the walls of the vessel in which the reaction was carried out. The process of Clark et al., is difficult to control, frequently leading to product degradation, and is virtually impossible to scale-up to larger production levels. The significant potential for product degradation is a direct consequence of maintaining the reaction mixture, a concentrated aqueous solution containing especially a frequently degradable protein as a component of the condensation adduct final product, as well as a starting material reactant, at elevated temperatures for extended periods of time. By contrast, the preparation processes of the present invention, especially the embodiments involving spray-drying and freeze-drying, reduce the residence time of the starting material reactants and condensation adduct final product in the aqueous solution to a minimum. Removal of the aqueous solvent by freeze-drying, i.e., lyophilization, is referred to by Clark et al., but their disclosure appears to indicate that the process was never attempted. The artisan could not, consequently, have any reasonable expectation of such a process working. Clark et al. do not suggest the criticality of using an o-hydroxy aldehyde and of maintaining the pH at 7.0 or above as in the process of the present invention. Even if the artisan were to carry out this lyophilization process of Clark et al., as with the evaporation process just described, it would be one characterized by prolonged desiccation, leading directly to the above-described process problems involving reproducibility, product quality and ability to scale up.

Nevertheless, the work of Clark et al. led to improved products which prior to that were sub-optimal with regard to long term storage stability and overall product purity, severely limiting their usefulness in treating animals. This had a particularly adverse impact in those cases where the product was a solid implant in pellet or related form for parenteral administration by instillation or insertion under the skin or within the muscle tissue of such an animal to be treated. While the method of Clark et al., was able to achieve some improvement in product stability, no practicable way was apparent by which to scale-up such a process for efficient and economically feasible larger scale commercial level production because of the large quantities of energy consumed and the long delays experienced in achieving complete evaporation of the aqueous solvent.

Accordingly, there still exists in the art a need to overcome the existing disadvantages of current processes of preparation for such Schiff base condensation adduct products, as well as to overcome the disadvantages of unstable adduct products produced by earlier, even less satisfactory processes. It is in the context of satisfying these needs in the art that the process of the present invention should be viewed.

In thus overcoming the disadvantages of the processes and products referred to in the technical literature, the gist of the present invention may be found in the discovery that removal of the aqueous solvent may be accomplished by methods which are very facile, reproducible and transposable, i.e., capable of being efficiently adapted to being carried out at a substantially larger scale, e.g., spray-drying, and which are therefore suitable for scaling up to manufacturing at commercial levels of efficient and economical production. An essential non-protein component of the Schiff base condensation adduct final product of the processes of preparation of the present invention is an aromatic o-hydroxy aldehyde of the type described in detail further below.

The Schiff base condensation adduct products were originally used in the art in an effort to overcome a problem relating to product stability in the basic protein involved. The cause of said problem is a direct result of the gradual denaturing of the protein product whereby there is a disruption of the tertiary structure, i.e., the configuration of the protein, and even some degeneration in aspects of the secondary and primary structure of said protein, resulting in an alteration of the physical properties of the protein and in a significant loss in the biological activity possessed by said protein. The Schiff base conjugation of such a protein and a carbonyl compound has been used in the art in an effort to achieve a more stable protein product, e.g., by the above-mentioned Clark et al., where it is alleged that a prolonged release form of somatotropin has been provided. The basic condensation reaction resulting in the formation of a Schiff base adduct is an equilibrium reaction which may be represented by the following schematic equation:

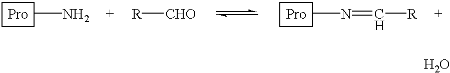

The fact that the Schiff base condensation reaction is one which is in equilibrium, and one which is not significantly shifted to the right in the above-depicted equation, is predictive of the problems which have arisen even with the solution proposed by Clark et al., namely, that significant amounts of the protein involved are not in the adduct form and therefore are subject to the disruption of their structure caused by denaturing with consequent loss of biological, in this case growth promoting, activity.

There is another aspect of the Schiff base condensation reaction which has posed a basic stumbling block to progress, especially as that method has been carried out by Clark et al. This problem involves sublimation of the aldehyde component, including aromatic o-hydroxy aldehydes, to a substantial degree during the long desiccation process. The extent of such sublimation can be as much as one-third or more of the total original aldehyde content of the reaction mixture. This significant loss of starting material reactant leads not only to the inevitable reduction in yield of final product, but to other problems as well, many of them created by unreacted protein starting material reactant. The unreacted protein is subject to degradation by denaturing during the desiccation process, and the resulting by-products add further process complications, e.g., precipitation on and adherence to the heat exchange surfaces of the processing apparatus described elsewhere herein. In contrast to these results, the processes of the present invention result in extremely high yields that almost entirely eliminate the problem of aldehyde component sublimation.

While removal of the water formed by condensation as shown on the right side of the above-depicted equation, would drive the reaction theoretically to completion in terms of the applicability of the law of mass action, such removal of the water of condensation becomes very problematical in view of the fact that the reaction is taking place in an aqueous solution. Removal of the water of condensation effectively means removal of all of the water present in the aqueous environment of the reaction. The art has now become aware of this inherent problem with Schiff base condensation adduct formation, but before the solution to this problem offered by the present invention, there has been no proposal put forward for its solution in the technical literature.

The first aspect of the present invention which is an essential element of its success is the use of an aromatic o-hydroxy aldehyde as one of the two key components reacted to form the Schiff base condensation adduct. The aromatic o-hydroxy aldehydes which are useful in the condensation process of the present invention preferably comprise one or more compounds of Formula (I):

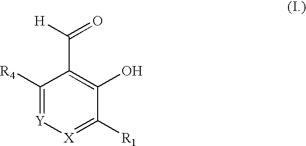

wherein:

R₁ and R₄ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂-C₆)alkenyl; —C(═O)OR₇ —OC(═O)R₇; —S(═O)₂; —S(═O)₂R₇; —S(═O)₂OR₇; —C(═O)NR₇R₉; —C(═O)R₉; —S(═O)₂N(R₇)(R₉); and —N(R₇)(R₉), where R₇ is hydrogen or (C₁-C₄)alkyl and R₉ is (C₁-C₄) alkyl;

wherein:

said alkyl, cycloalkyl and alkenyl groups defining R₁ and R₄ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl; and

X and Y are independently N, O, S, CHR₂, or CHR₃, respectively, provided that X and Y may not both be selected from O and S at the same time;

where

R₂ and R₃ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂-C₆)alkenyl; —C(═O)OR₁₁ —OC(═O)R₁₁; —S(═O)₂; —S(═O)₂N(R₁₁)(R₁₃); and —N(R₁₁)(R₁₃),

where

R₁₁ is hydrogen or (C₁-C₄)alkyl and R₁₃ is (C₁-C₄)alkyl; and

wherein

said alkyl, cycloalkyl and alkenyl groups defining R₂ and R₃ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl.

In accordance with a preferred aspect of the present invention, R₁ and R₄ are independently selected from the group consisting of hydrogen; hydroxy; trifluoromethyl; (C₁-C₄)alkyl; (C₁-C₄)alkoxy; —C(═O)OR₇ and —N(R₇)(R₉), where R₇ is hydrogen or (C₁-C₂)alkyl and R₉ is (C₁-C₂).

In more preferred embodiments of the present invention, R₁ and R₄ are independently selected from the group consisting of hydrogen; hydroxy; (C₁-C₂)alkyl; (C₁-C₂)alkoxy; carboxyl and methylamino. In this particular embodiment, R₇ is hydrogen and R₉ is methyl. It is also preferred that when R₁ and R₄ are defined as alkyl and are substituted, that there be a single substituent selected from the group consisting of hydroxy; (C₁-C₂)alkoxy; carboxyl; amino disubstituted by (C₁-C₂)alkyl; and sulfonamido disubstituted by (C₁-C₂)alkyl. Even more preferably still, said single substituent is selected from the group consisting of hydroxy, methoxy, and dimethylamino.

Further, in preferred aspects of the present invention, X and Y are independently selected from the group consisting of N, CHR₂, or CHR₃; and in more preferred aspects of the present invention, one of X or Y is N and the other is CHR₂, or CHR₃, respectively. More preferably still in the present invention X is CHR₂ and Y is CHR₃, wherein R₂ and R₃ are preferably independently selected from the group consisting of hydrogen; hydroxy; halo; trifluoromethyl; (C₁-C₄)alkyl; (C₁-C₄)alkoxy; —C(═O)OR₁₁ —S(═O)₂N(R₁₁)(R₁₃); and —N(R₁₁)(R₁₃), where R₁₁ is preferably hydrogen or (C₁-C₂)alkyl and R₁₃ is (C₁-C₂)alkyl. Even more preferably still in the present invention R₂ and R₃ are independently selected from the group consisting of hydrogen; hydroxy; C₁-C₂)alkyl; (C₁-C₂)alkoxy; carboxyl; and methylamino. In the latter case R₁₁ is hydrogen and R₁₃ is methyl.

It is preferred in the present invention that when R₂ and R₃ are defined as alkyl and are substituted, that there be a single substituent selected from the group consisting of hydroxy; (C₁-C₂)alkoxy; carboxyl; amino disubstituted by (C₁-C₂)alkyl; and sulfonamido disubstituted by (C₁-C₂)alkyl.

In order to further illustrate this aspect of the present invention relating to the particular aromatic o-hydroxy aldehydes which are especially suitable for use therein, there follows immediately below tables of groups of such preferred aldehydes:

(I)

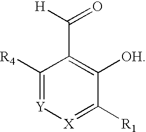

| R₁ | R₄ | X | Y | R₂ | R₃ |
|---|---|---|---|---|---|
| H | H | CHR₂ | CHR₃ | H | H |
| H | OH | CHR₂ | CHR₃ | H | H |
| OH | H | CHR₂ | CHR₃ | H | H |
| CF₃ | H | CHR₂ | CHR₃ | H | H |
| CH₃ | H | CHR₂ | CHR₃ | H | H |
| CH₂CH₃ | H | CHR₂ | CHR₃ | H | H |
| OCH₃ | H | CHR₂ | CHR₃ | H | H |
| C(═O)OH | H | CHR₂ | CHR₃ | H | H |
| C(═O)OCH₃ | H | CHR₂ | CHR₃ | H | H |
| NHCH₃ | H | CHR₂ | CHR₃ | H | H |
| N(CH₃)₂ | H | CHR₂ | CHR₃ | H | H |
| H | OH | CHR₂ | CHR₃ | H | H |
| H | CH₃ | CHR₂ | CHR₃ | H | H |
| H | CF₃ | CHR₂ | CHR₃ | H | H |
| H | CH₂CH₃ | CHR₂ | CHR₃ | H | H |
| H | OCH₃ | CHR₂ | CHR₃ | H | H |
| H | C(═O)OH | CHR₂ | CHR₃ | H | H |
| H | C(═O)OCH₃ | CHR₂ | CHR₃ | H | H |
| H | NHCH₃ | CHR₂ | CHR₃ | H | H |
| H | N(CH₃)₂ | CHR₂ | CHR₃ | H | H |
| OH | OH | CHR₂ | CHR₃ | H | H |
| CF₃ | CF₃ | CHR₂ | CHR₃ | H | H |
| CH₃ | CH₃ | CHR₂ | CHR₃ | H | H |
| CH₂CH₃ | CH₂CH₃ | CHR₂ | CHR₃ | H | H |
| OCH₃ | OCH₃ | CHR₂ | CHR₃ | H | H |
| C(═O)OH | C(═O)OH | CHR₂ | CHR₃ | H | H |
| C(═O)OCH₃ | C(═O)OCH₃ | CHR₂ | CHR₃ | H | H |
| NHCH₃ | NHCH₃ | CHR₂ | CHR₃ | H | H |
| N(CH₃)₂ | N(CH₃)₂ | CHR₂ | CHR₃ | H | H |
| H | H | CHR₂ | CHR₃ | OH | H |
| H | H | CHR₂ | CHR₃ | H | OH |
| H | H | CHR₂ | CHR₃ | OH | OH |
| H | H | CHR₂ | CHR₃ | CH₃ | H |
| H | H | CHR₂ | CHR₃ | H | CH₃ |
| H | H | CHR₂ | CHR₃ | CH₃ | CH₃ |
| H | H | CHR₂ | CHR₃ | OCH₃ | H |
| H | H | CHR₂ | CHR₃ | H | OCH₃ |
| H | H | CHR₂ | CHR₃ | OCH₃ | OCH₃ |
| H | H | CHR₂ | CHR₃ | NHCH₃ | H |
| H | H | CHR₂ | CHR₃ | H | NHCH₃ |
| H | H | CHR₂ | CHR₃ | NHCH₃ | NHCH₃ |
| H | H | CHR₂ | CHR₃ | N(CH₃)₂ | H |
| H | H | CHR₂ | CHR₃ | H | N(CH₃)₂ |
| H | H | CHR₂ | CHR₃ | N(CH₃)₂ | N(CH₃)₂ |
| CH₃ | H | CHR₂ | CHR₃ | CH₃ | H |
| H | CH₃ | CHR₂ | CHR₃ | H | CH₃ |
| OCH₃ | H | CHR₂ | CHR₃ | OCH₃ | H |
| OCH₃ | H | CHR₂ | CHR₃ | H | CH₃ |
| H | H | CHR₂ | CHR₃ | H | OH |
| H | OH | CHR₂ | CHR₃ | CH₃ | CH₃ |
| OCH₃ | H | CHR₂ | CHR₃ | OCH₃ | H |
| OH | H | CHR₂ | CHR₃ | OCH₃ | OCH₃ |
| OCH₃ | H | CHR₂ | CHR₃ | H | NHCH₃ |
| H | NHCH₃ | CHR₂ | CHR₃ | NHCH₃ | H |
| H | OH | CHR₂ | CHR₃ | H | NHCH₃ |
| H | OH | CHR₂ | CHR₃ | OH | H |
| H | OH | CHR₂ | CHR₃ | H | OH |
| N(CH₃)₂ | H | CHR₂ | CHR₃ | OCH₃ | H |
| CH₃ | H | CHR₂ | CHR₃ | H | OCH₃ |
| H | CH₃ | CHR₂ | CHR₃ | N(CH₃)₂ | H |
| H | N(CH₃)₂ | CHR₂ | CHR₃ | CH₃ | H |
| OCH₃ | H | CHR₂ | CHR₃ | H | OCH₃ |
| OCH₃ | H | CHR₂ | CHR₃ | CH₃ | CH₃ |
| OCH₃ | H | O | CHR₃ | — | H |
| H | OCH₃ | O | CHR₃ | — | H |
| CH₃ | H | O | CHR₃ | — | CH₃ |
| H | OCH₃ | CHR₂ | O | H | CH₃ |
| H | H | CHR₂ | O | N(CH₃)₂ | — |
| H | CH₃ | CHR₂ | O | CH₃ | — |
| CH₃ | H | S | CHR₃ | — | CH₃ |
| OCH₃ | H | S | CHR₃ | — | H |
| N(CH₃)₂ | H | CHR₂ | S | H | — |
| OCH₃ | H | CHR₂ | S | OCH₃ | — |
| OCH₃ | H | N | CHR₃ | — | H |
| CH₃ | H | N | CHR₃ | — | CH₃ |
| H | N(CH₃)₂ | N | CHR₃ | — | H |
| H | CH₃ | N | CHR₃ | — | CH₃ |
| OCH₃ | OCH₃ | N | CHR₃ | — | H |
| CH₃ | H | N | CHR₃ | — | NHCH₃ |
| CH₃ | OCH₃ | N | CHR₃ | — | H |
| CH₃ | CH₂OH | N | CHR₃ | — | H |
| CH₃ | CH₂OH | N | CHR₃ | — | CH₃ |
| OCH₃ | CH₂OH | N | CHR₃ | — | H |
| OCH₃ | CH₃ | CHR₂ | N | H | — |
| NHCH₃ | H | CHR₂ | N | H | — |
| H | CH₃ | CHR₂ | N | CH₃ | — |
| H | H | CHR₂ | N | N(CH₂)₂ | — |
| CH₃ | CH₂OH | CHR₂ | N | H | — |
| OCH₃ | CH₂OH | CHR₂ | N | H | — |
| CH₃ | CH₂OH | CHR₂ | N | CH₃ | — |
| CH₃ | CH₃ | N | N | — | — |
| CH₃ | CH₂OH | N | N | — | — |
| OCH₃ | OCH₃ | N | N | — | — |

From among the above-recited species of aromatic o-hydroxy aldehydes which are suitable and preferred for use in the preparation processes and products thereof of the present invention, there are several which are most preferred for such use based on their availability, cost, effectiveness, and facility and efficiency in said processes, and based on their ability to produce an improved product which possesses the optimal desired characteristics in terms of stability over time and maintenance of the original levels of biological activity. Said most preferred species comprise salicylaldehyde; 2,3-dihydroxybenzaldehyde; 2,6-dihydroxybenzaldehyde; o-vanillin; and pyridoxal; which may be represented by the following structural formulas:

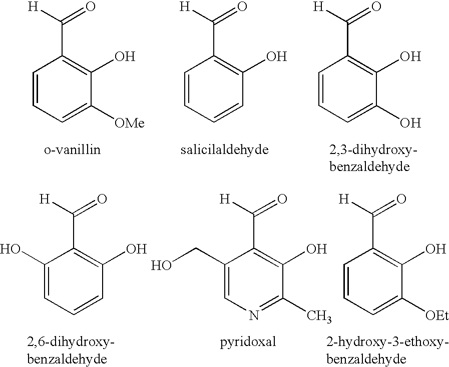

There has been described immediately above the first aspect of the present invention which is an essential element of its success, i.e., the use of an aromatic o-hydroxy aldehyde as one of the two key components reacted to form the improved Schiff base condensation adduct. There below follows, accordingly, a detailed description of the second component, a protein, which is reacted with said aromatic o-hydroxy aldehyde in accordance with the procedures of the preparation process of the present invention, to form the improved Schiff base condensation adduct final products of the present invention.

A protein which is a candidate for use as the second component of the improved Schiff base condensation adduct of the present invention must meet several requirements before it is judged suitable for such use. First, there is no sharply defined limitation which can be based solely on the size of the protein and nothing else. The molecular weight or mass of the protein is expressed in Daltons or kiloDaltons (kDs), and it may be comprised of from as little as two amino acids up to several hundred to as many as a thousand or more amino acids. A typical protein has a mass of 30,000 Daltons. It is important, however, that the candidate protein be such that it can be administered to an animal and thereafter be taken up, beneficially utilized, metabolized and cleared, i.e., eliminated from said animal. It is desirable that said protein candidate have such characteristics before it is reacted with an aromatic o-hydroxy aldehyde to form the improved Schiff base condensation adducts of the present invention; nevertheless, the formation of such a condensation adduct will significantly enhance such properties and characteristics and may thereby render suitable a protein candidate that would otherwise fail to be suitable.

The other primary characteristic of a protein candidate suitable for use in forming the Schiff base condensation adducts of the present invention is its ability to achieve a beneficial utility in the particular animal or animals to which it is administered. This beneficial utility is most commonly one which is therapeutic, whether administered to animals or humans. As used herein the terms “animal” and “animals” refer to all members of the animal kingdom and the primary divisions thereof which satisfy the other requirements imposed by the present invention with regard to proteins having beneficial utility with respect thereto. The expression “beneficial utility” as used herein usually denotes activity of benefit to the particular animal, and therefore to humans in terms of the economic rewards of animal husbandry. However, this expression also extends to activity which is disadvantageous or detrimental to the particular animal, but may, conversely, be of economic advantage to humans. Such activity would include pesticidal activity of various kinds, e.g., inhibition of growth and reproduction or outright destruction of pests which damage crops of economic importance or injure domesticated animals of value to humans. Accordingly, all of the major phyla and subdivions thereof which are of economic significance are included within the scope of the present invention, e.g., vertebrates of the phylum Arthropoda which includes classes of insects (Insecta), spiders and mites (Arachnida), and crustaceans (Crustacea); or of the subphylum Vertebrata which includes classes of mammals (Mammalia), birds reptiles (Reptilia), amphibians (Amphibia) and fishes; and invertebrates of the phylum Mollusca which includes clams and snails; or of the phylum Annelida, which includes earthworms and leeches; or of the phylum Echinodermata, which includes starfishes and sea urchins; or of the phylum Nematoda, which includes heartworms.

Proteins suitable for use in the present invention can have still other biological activities which are of benefit to an animal or to the use of an animal which would not usually be classified as therapeutic in nature. For example, protein hormones such as somatotropin are used to regulate the growth of animals usually kept as domestic stock for food production, and somatotropin administered to such animals has the beneficial utility of increasing feed utilization efficiency and reducing the time necessary to bring such a stock animal to market. Use of such a protein hormone has a clear and definite commercial and economic benefit not directly related to therapy as such.

Other hormones and regulators of body functions which are proteins and are currently being commercially exploited may also be improved by use of the processes of the present invention. Such improvement is with respect to their improved level of adduct condensation as well as with respect to enhanced yields thereof, which results in improved opportunities for their administration to animals by such modes as parenteral solid implants.

However, the great majority of proteins which may be used with the processes of the present invention are those which have recognized utility as therapeutic agents for animals and man. These proteins have found use or have been actively explored for use in a wide variety of therapeutic classes. The description in the below paragraphs makes clear that not only are there a large number of such proteins, but that all of these proteins benefit from improved long term stability and preservation of their biological activity when prepared in accordance with the process of the present invention as improved Schiff base condensation adducts. This is particularly true where the proteins are prepared in pellet or similar form to be used as an implanted depot for sustained release administration.

There is a large group of proteinaceous endogenous and synthetic opioid analgesics and antagonists which have been organized into three distinct families identified as the enkephalins, the endorphins, and the dynorphins. These proteins are selective and nonselective agonists and antagonists of the μ, κ, and δ opioid receptor subtypes, with therapeutic utility primarily as analgesics. Specific proteins include [Leu⁵] and [Met⁵]enkephalin; dynorphin A and B; α- and β-neoendorphin; [D-Ala², MePhe⁴,-Gly(ol)⁵]enkephalin (DAMGO); [D-Pen², D-Pen⁵]enkephalin (DPDPE); [D-Ser², Leu⁵]enkephalin-Thr⁶ (DSLET); [D-Ala², D-Leu⁵]enkephalin (DADL); D-Phe-Cys-Tyr-D-Trp-Orn-Thr-Pen-Thr-NH₂ (CTOP); [D-Ala², N-MePhe⁴, Met(O)⁵-ol]enkephalin (FK-33824); Tyr-D-Ala-Phe-Asp-Val-Val-Gly-NH₂ ([D-Ala²]deltorphin I; Tyr-D-Ala-Phe-Glu-Val-Val-Gly-NH₂ ([D-Ala², Glu⁴]deltorphin II; Tyr-Pro-Phe-Pro-NH₂ (morphiceptin); Tyr-Pro-MePhe-D-Pro-NH₂ (PL-017); and [D-Ala², Leu⁵, Cys⁶]enkephalin.

A group of proteins classified as autocoids which includes bradykinin and kallidin is produced by a series of proteolytic reactions in response to inflammatory events such as tissue damage, viral infections, and allergic reactions. These proteins act locally and produce pain, vasodilatation, increased vascular permeability and the synthesis of prostaglandins. These proteins and their analogous derivatives having agonist and antagonist activity are potentially useful therapeutic agents for the treatment of male infertility, for the delivery of cancer chemotherapeutic agents beyond the blood-brain barrier, and for the treatment of pain, asthma, and other chronic inflammatory diseases. Specific proteins of this type include: Arg-Pro-Pro-Gly-Phe-Ser-Pro-Phe-Arg (bradykinin); Lys-Arg-Pro-Pro-Gly-Phe-Ser-Pro-Phe-Arg (kallidin); Arg-Pro-Pro-Gly-Phe-Ser-Pro-Phe (des-Arg⁹-bradykinin); Lys-Arg-Pro-Pro-Gly-Phe-Ser-Pro-Phe (des-Arg¹⁰-kallidin); Arg-Pro-Pro-Gly-Phe-Ser-Pro-Leu (des-Arg⁹-[Leu⁸]-bradykinin); Arg-Pro-Pro-Gly-Phe-Ser-[D-Phe]-Phe-Arg ([D-Phe⁷]-bradykinin); and [D-Arg]-Arg-Pro-Hyp-Gly-Thi-Ser-Tic-Oic-Arg (HOE 140), where Hyp is trans4-hydroxy-Pro; Thi is β-(2-thienyl)-Ala; Tic is [D]-1,2,3,4-tetrahydroquinolin-3-yl-carbonyl; and Oic is (3 as, 7 as)-octahydroindol-2-yl-carbonyl.

Regulation of body fluid osmolality by vasopressin and related agents affecting the renal conservation of water and the creation of analogues of such proteins which are selective for the vasopressin receptor subtypes V₁ and V₂ which mediate pressor responses and antidiuretic responses, respectively, have led to a number of therapeutic agents with different activities. For example V₁ antagonists may be beneficial in the treatment of congestive heart failure, hypertension, and postoperative ileus and abdominal distension. V₂ agonists may be used to treat central diabetes insipidus by controlling polyuria and polydipsia, and to treat bleeding disorders such as von Willebrand's disease. Specific naturally-occurring vasopressin-like peptides include arginine vasopressin (AVP) of the following formula:

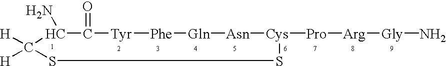

and lypressin ([Lys⁸]-AVP; synthetic vasopressin peptides: V₁ₐ-selective agonist [Phe², Ile², Orn⁸]AVP; V_{1b}-selective agonist deamino [D-3-(3′-pyridyl)-Ala²]AVP; V₂-selective agonists desmopressin (dDAVP), and deamino[Val⁴, D-Arg⁸]AVP; and peptide antagonists such as V₁ₐ-selective antagonist d(CH₂)₅[Tyr(Me)²]AVP of the following formula:

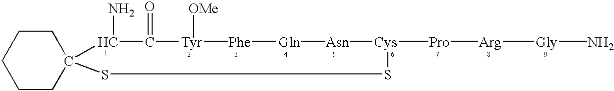

as well as V_{1b}-selective antagonist dp[Tyr(Me)²]AVP; and V₂-selective antagonists des Gly-NH₂⁹-d(CH₂)₅[D-Ile², Ile⁴]AVP, and d(CH₂)₅[D-Ile², Ile⁴, Ala-N H₂⁹]AVP.

Pentagastrin is a protein diagnostic aid used as an indicator of gastric secretion and has the following formula: N-t-butyloxycarbonyl-β-Ala-Trp-Met-Asp-Phe-NH₂.

Octreotide is a synthetic analog of somatotstatin and is useful in treating the symptoms of tumors of the gastrointestinal tract, diarrhea refractory to other treatment, various motility disorders, and gastrointestinal bleeding. Octreotide, available as the acetate and pamoate, has the structure: L-cysteinamide-D-Phe-L-Cys-L-Phe-D-Trp-L-Lys-L-Thr-N-[2-hydroxy-1-(hydroxymethyl)propyl]-. cyclic (2→7)-disulfide, [R—(R*,R*)]—.

A number of antibody reagents for use as immunosuppressive agents have been approved for clinical use. Advanced hybridoma technology permits the production of such antibodies in large quantities from continuously cultured cells which generate highly purified and specific antibody preparations that can be used as standardized pharmacological reagents. Such antibody reagents include antithymocyte globulin; muromonab-CD3 monoclonal antibody; and Rh₀(D) immune globulin. Protein immunostimulants which have been developed for therapeutic use in treating immunodeficiency states includes immune globulin.

The cytokines are a group of diverse proteins produced by leukocytes and related cells which have a variety of immunoregulatory effects. The major currently recognized cytokines consist of interferons, colony-stimulating factors, and interleukins. Specific examples of members of these classes include α-interferon; interferon-γ (IFN-γ); granulocyte colony-stimulating factor (G-CSF); granulocyte macrophage colony-stimulating factor (GM-CSF); and interleukin-1 (IL-1) through interleukin-12 (IL-12).

Hematopoietic growth factors are a group of hormonelike glycoproteins involved in the regulation of the process whereby mature blood cells are continuously replaced. Clinical applications of these proteins include treatment of primary hematological diseases and uses as adjunctive agents in the treatment of severe infections and in the management of patients who are undergoing chemotherapy or marrow transplantation. Specific examples of such growth factors include erythropoietin (EPO); stem cell factor (SCF); interleukins (IL-1-12); monocyte/macrophage colony-stimulating factor (M-CSF, CSF-1); P1XY321 (GM-CSF/IL-3 fusion protein); and thrombopoietin.

Thrombolytic drugs are used to dissolve both pathological thrombi and fibrin deposits at sites of vascular injury, and include such proteins as streptokinase; tissue plasminogen activator (t-PA); and urokinase.

Anterior pituitary hormones and the hypothalamic factors that regulate their use are proteins having therapeutic uses. The anterior pituitary hormones are divided into three classes: (a) somatotropic hormones which include growth hormone (GH), prolactin (Prl), and placental lactogen (PL); (b) glycoprotein hormones which include luteinizing hormone (LH), follicle-stimulating hormone (FSH), and thyroid-stimulating hormone (TSH); and (c) POMC-derived hormones which include corticotropin (ACTH), α-melanocyte-stimulating hormone (α-MSH), β-melanocyte-stimulating hormone (β-MSH), β-lipotropin (β-LPH), and γ-lipotropin (γ-LPH). The hypothalamic factors which regulate release of said hormones include growth hormone-releasing hormone (GHRH), luteininzing hormone releasing hormone (LHRH), insulin-like growth factor (IGF-1 and IGF-2), somatostatin, and gonadotropin-releasing hormone (GnRH).

Growth hormone is used as replacement therapy in growth-hormone deficient children. Somatostatin is a hypothalamic substance which inhibits growth hormone release but has a short half-life. Its synthetic analogue, octreotide, already described further above, is used to treat acromegaly caused by excessive secretion of growth hormone. The gonadotropic hormones include LH, FSH, and corionic gonadotropin (GC) and are used diagnostically. CG is used to detect pregnancy, while LH and FSH are used in the diagnoses of several reproductive disorders. These protein gonadotropins are also used therapeutically in the treatment of infertility. For example, urofollitropin is a human menopausal gonadotropin (hMG) or menotropin preparation from which most of the LH has been removed, and is thus primarily FSH. Urofollitropin is used to induce ovulation. Gonadorelin is a preparation of synthetic human GnRH which is used therapeutically to stimulate gonadotropin secretion. On the other hand, synthetic GnRH agonists, e.g., leuprolide, histrelin, nafarelin, and goserelin may be used to treat a variety of endocrine disorders that are responsive to reductions in gonadal steroids.

Thyroid function is regulated by thyrotropin (TSH), a glycoprotein, the secretion of which is controlled by thyrotropin-releasing hormone (TRH). Therapeutic use of TSH is for hormone replacement therapy in patients with hypothyroidism and for TSH suppression therapy in patients with nontoxic goiter or after treatment for thyroid cancer.

The protein insulin is the mainstay for treatment of virtually all insulin-dependent diabetes mellitus (IDDM) patients and many non-insulin-dependent diabetes mellitus (NIDDM) patients. Synthetic analogs of insulin are also used which are more rapidly absorbed from subcutaneous sites. Also, implantable pellets have been designed to release insulin slowly over days or weeks. Glucagon is a protein which has a significant physiological role in the regulation of glucose and ketone body metabolism, and is used to treat severe hypoglycemia, and is also used by radiologists for its inhibitory effects on the gastrointestinal tract. Somatostatin, referred to further above, is a hormone with a short biological half life which has limited its used mainly to blocking hormone release in endocrine-secreting tumors, including insulinomas, glucagonomas, VIPomas, carcinoid tumors, and somatotropinomas. The synthetic analogue, octreotide, is longer-acting and consequently more frequently used for therapeutic treatment.

Calcitonin (CT) is a hormone which acts specifically on osteoclasts to inhibit bone resorption and is useful in managing hypercalcemia. CT is also effective in disorders of increased skeletal remodeling, such as Paget's disease. The protein parathyroid hormone (PTH) is of potential value in the treatment of patients with spinal osteoporosis.

In addition to the above-described classes of protein therapeutic agents, the below enumerated species of protein compositions have been approved for use in humans.

Aldesleukin, 125-L-serine-2-133-interleukin 2, is used as an antineoplastic agent and as an immunostimulant.

Alglucerase is a monomeric glycoprotein of 497 amino acids that is a modified form of human placental tissue β-glucocerebrosidase, and is used as a replenisher of the glucocerebrosidase enzyme.

Alsactide is a synthetic corticotropin analogue, 1-β-Ala-17[L-2,6-diamino-N-(4-aminobutyl)hexanamide]-α¹⁻¹⁷-corticotropin.

Alteplase is a serine protease of 527 amino acids whose sequence is identical to the naturally occurring protease produced by endothelial cells in vessel walls, and which is used as a plasminogen activator.

Alvircept sudotox is a synthetic chimeric protein engineered to link the first 178 amino acids of the extracellular domain of CD₄ via two linker residues to amino acids 1-3 and 253-613 of Pseudomonas exotoxin A, and which is used as an antiviral agent.

Amlintide is a protein of 37 amino acids which is used as an antidiabetic agent.

Amogastrin is N-carboxy-L-Trp-L-Met-L-α-Asp-3-phenyl-L-Alaninamide.

Anakinra is N²-L-Met-interleukin 1 receptor antagonist used as a nonsteroidal anti-inflammatory and as a suppressant for treating inflammatory bowel disease.

Anaratide acetate is atriopeptin-21 (rat), N-L-Arg-8-L-Met-21a-L-Phe-21b-L-Arg-21c-L-Tyr-, acetate, which is used as an antihypertensive agent and as a diuretic.

Angiotensin amide is angiotensin II, 1-L-Asn-5-L-Val-, which is used as a vasoconstrictor.

Aprotinin is a pancreatic trypsin inhibitor having 58 amino acids which is used as an enzyme inhibitor (proteinase).

Arfalasin is 1-succinamic acid-5-L-Val-8-(L-2-phenylglycine)angiotensin II which is used as an antihypertensive agent.

Argipressin tannate is vasopressin, 8-L-Arg-, tannate, which is used as an antidiuretic.

Aspartocin is an antibiotic agent produced by Streptomyces griseus, and is oxytocin, 4-L-Asn-.

Atosiban is oxytocin, 1-(3-mercaptopropanoic acid)-2-(O-ethyl-D-Tyr)₄-L-Thr-8-L-Orn-, which is used as an oxytocin antagonist

Avoparcin is a glycopeptide antibiotic obtained from Streptomyces candidus.

Basifungin is an antifungal agent having the structure N-[(2R,3R)-2-hydroxy-3-MeVal]-N-L-MeVal-L-Phe-N-L-MePhe-L-Pro-L-allo-Ile-N-L-MeVal-L-Leu-3-hydroxy-N-L-MeVal α₁-lactone.

Becaplermin is recombinant human platelet-derived growth factor B, which is a recombinant protein produced by genetically engineered Saccharomyces cerevisiae that is similar in amino acid composition and biological activity to the endogenous human PDGF-BB homodimer, which is used for the treatment of chronic dermal ulcers by promoting the proliferation of mesenchymally-derived cells.

Bivalirudin is an anticoagulant, antithrombotic agent having 20 amino acids.

Carbetocin is 1-butyric acid-2-[3-(p-methoxyphenyl)-L-Ala]oxytocin.

Cargutocin is 1-butyric acid-6-(L-2-aminobutyric acid)-7-glycineoxytocin.

Ceruletide is a gastric secretory stimulant of the structure 5-O-L-Pro-L-Gln-L-α-Asp-L-O-sulfo-L-Tyr-L-Thr-L-Gly-L-Trp-L-Met-L-α-Asp-L-Phe-amide.

Cetermin is transforming human growth factor β2 having 112 amino acids.

Cilmostim is 1-233-colony-stimulating factor 1 (human clone p3ACSF-69 protein moiety), cyclic (7→90), (48→139), (102→146)-tris(disulfide) dimer used as a hematopoietic agent (macrophage colony-stimulating factor).

Colistimethate sodium is a colistin A component useful as an antibacterial agent.

Corticorelin Ovine Triflutate is corticotropin-releasing factor (sheep), trifluoroacetate salt, which is used as a diagnostic aid for adrenocortical insufficiency and Cushing's syndrome, and as a corticotropin-releasing hormone.

Cosyntropin is tetracosactide acetate, α¹⁻²⁴-corticotropin, which is used as an adrenocorticotropic hormone.

Cyclosporin is a cyclic protein containing 11 amino acids and a 3-hydroxy4-methyl-2-(methylamino)-6-octenoyl moiety at the 6-position, which is used as an immunosuppressant.

Dacliximab (Ro-24-7375) is a humanized anti-TAC monoclonal antibody comprised of four subunits linked via disulfide bridges and a molecular weight of approximately 150 kD, which is used as an immunosuppressant. A similar immunosuppressant protein is daclizumab.

Daptomycin is a proteinaceous antibacterial agent.

Desirudin is 63-desulfohirudin from Hirudo medicinalis comprising 63 amino acids, which is used as an anticoagulant.

Deslorelin is luteinizing hormone-releasing factor (pig) comprising 9 amino acids, which is used as an LHRH agonist.

Desmopressin acetate is vasopressin, 1-(3-mercaptopropanoic acid)-8-D-Arg-, monoacetate salt, trihydrate, comprising 9 amino acids, which is used as an antidiuretic.

Detirelix acetate comprises 10 amino acids and is used as an LHRH antagonist.

Dumorelin is 27-L-Leu-44a-Gly growth hormone-releasing factor (human).

Elcatonin is 1-butyric acid-7-(L-2-aminobutyric acid)-26-L-Asp-27-L-Val-29-L-Ala calcitonin (salmon).

Emoctakin is interleukin 8 (human) comprising 72 amino acids with two Cys bridges.

Epoetin alfa is a 165 amino acid glycoprotein that regulates red blood cell production and is produced by Chinese hamster ovary cells into which the human erythropoietin gene has been inserted. It is used as an anti-anemic and hematinic agent.

Ersofermin is recombinant human basic fibroblast growth factor (bFGF) comprising 157 amino acids, a non-glycosylated protein isolated from human placenta and cloned and expressed in E. coli. It is used as a wound healing agent.

Felypressin is vasopressin, 2-L-Phe-8-L-Lys comprising 9 amino acids, which is used as a vasoconstrictor.

Filgrastim is a single chain 175 amino acid polypeptide, which is non-glycosylated and expressed by E. coli, and which is used as an antineutropenic agent and as a haematopoietic stimulant.

Glucagon is a single chain protein of 29 amino acids which is used an antidiabetic agent.

Gonadorelin acetate is the diacetate salt of luteininzing hormone-releasing factor acetate comprising 10 amino acids, which is used as a gonad-stimulating principle.

Goserelin is luteinizing hormone-releasing factor (pig) comprising 9 amino acids, which is used as an LHRH agonist.

Histrelin is luteinizing hormone-releasing factor (pig) comprising 9 amino acids, which is used as an LHRH agonist.

Imiglucerase is 495-L-Histidineglucosylceramidase placenta isoenzyme protein, which is used as an enzyme replenisher for glucocerebrosidase.

Insulin, Dalanated is an insulin derivative prepared by removal of the C-terminal alanine from the B chain of insulin, which is used as an antidiabetic agent.

Interferon alfa-2a is interferon αA (human leukocyte protein moiety reduced) comprising 165 amino acids, which is used as an antineoplastic agent and as a biological response modifier. Interferon alfa-2b is interferon α2b (human leukocyte clone Hif-SN206 protein moiety reduced) comprising 165 amino acids, which is also used as an antineoplastic agent and as a biological response modifier. Interferon beta-1a is a glycosylated polypeptide consisting of 166 amino acid residues produced from cultured Chinese hamster ovary cells containing the engineered gene for human interferon beta, which is also used as an antineoplastic agent and as a biological response modifier. Interferon beta-1b is a non-glycosylated polypeptide consisting of 165 amino acid residues produced from E. coli, which is also used as an immunomodulator. Interferon gamma-1b is 1-139 interferon γ (human lymphocyte protein moiety reduced), N²-L-Met, which is used as an antineoplastic agent and as an immunomodulator.

Iroplact is N-methionylblood platelet factor 4 (human subunit) comprising 71 amino acid residues having two Cys bridges.

Lanoteplase is a tissue plasminogen activator protein derived from human t-PA by deletion of the fibronectin-like and the EGF-like domains and mutation of Asn 117 to Gln 117. The protein is produced by expression in a mammalian host cell of a DNA sequence encoding the peptide sequence, and the protein is used as a plasminogen activator and thrombolytic agent.

Lanreotide acetate comprises 8 amino acids and has one disulfide bridge. The protein is used as an antineoplastic agent.

Lenograstim is a glycoprotein consisting of 174 amino acid residues which is produced in Chinese hamster ovary cells by expression of a human granulocyte colony-stimulating factor-cDNA derived from a human oral cavity squamous cell line-mRNA. The protein is used as an antineutropenic agent and as an haematopoietic stimulant.

Lutrelin acetate is a luteinizing hormone-releasing factor (pig) comprising 9 amino acids, which is used as an LHRH agonist.

Molgramostim is a colony-stimulating factor 2 (human clone pHG₂₅ protein moiety reduced) comprising 127 amino acids, which is used as an antineutropenic agent and as an haematopoietic stimulant.

Murodermin is an epidermal growth factor (mouse salivary gland).

Nafarelin acetate is luteinizing hormone-releasing factor (pig) comprising 9 amino acids, which is used as an LHRH agonist.

Nagrestipen is 26-L-Alaninelymphokine MiP 1α (human clone pAT 464 macrophage inflammatory comprising 69 amino acids and having two disulfide bridges.

Pepstatin is N-(3-methyl-1-oxobutyl)-L-Val-L-Val-4-amino-3-hydroxy-6-methylheptanoyl-L-Ala-4-amino-3-hydroxy-6-methylheptanoic acid, which is used as a pepsin enzyme inhibitor.

Pramlintide is a protein comprising 37 amino acids and having one disulfide bridge, which is used as an antidiabetic agent.

Proinsulin Human is proinsulin (pig) comprising 86 amino acid residues and having three disulfide bridges, which is used as an antidiabetic agent.

Sargramostim is colony-stimulating factor 2 (human clone pHG25 protein moiety), 23-L-Leu-, a single chain, glycosylated polypeptide of 127 amino acid residues expressed from Saccharomyces cerevisiae, which is used as an antineutropenic agent and a haematopoietic stimulant.

Somagrebove is somatotropin (ox reduced), 1-[N²-L-Met-L-α-Asp-L-Glutamine]- comprising 191 amino acids, which is used as a galactopoietic agent especially for veterinary use.

Somalapor is somatotropin (pig clone pPGH-1 reduced), N-L-Alanyl-growth hormone comprising a total of 191 amino acids, which is used as a hormone (growth, porcine).

Somatrem is somatotropin (human), N-L-Met- comprising 191 amino acids having two disulfide bridges, which is used as a growth hormone.

Somatotropin, which is also sometimes referred to as adenohypophseal growth hormone, GH, hypophyseal growth hormone, anterior pituitary growth hormone, pituitary growth hormone, and somatotropic growth hormone, is a species specific anabolic protein which promotes somatic growth, stimulates protein synthesis, and regulates carbohydrate and lipid metabolism. Somatotropin is secreted by the anterior pituitary under the regulation of the hypothalamic hormones somatoliberin and somatostatin. Somatotropin growth hormones from various species differs in amino acid sequence, antigenicity, isoelectric point, and in the range of animals in which they can produce biological responses.

In humans somatotropin is a single polypeptide chain comprising 191 amino acids having the normal structure of the principal growth stimulating hormone obtained from the anterior lobe of the human pituitary gland, which is used as a growth hormone. Somatotropin is also available in recombinant form. As used herein, the term “somatotropin” is intended to include naturally occurring as well as synthetically, including recombinantly derived human and animal somatotropins (growth hormones), especially bovine and porcine somatotropins. Methionyl human growth hormone, C₉₉₅H₁₅₃₇N₂₆₃O₃₀₁S₈, is produced in bacteria from recombinant DNA, and contains the complete amino acid sequence of the natural hormone plus an additional N-terminal methione.

There are four naturally occurring molecular variants of bovine somatotropin, one of which is known as somavubove. Several variants have been produced by recombinant DNA technology, including somagrebove, C₉₈₇H₁₅₅₀N₂₆₈O₂₉₁S₉; sometribove, C₉₇₈H₁₅₃₇N₂₆₅O₂₈₆S₉ and somidobove, C₁₀₂₀H₁₅₉₆N₂₇₄O₃₀₂S₉.

Several variants of naturally-occurring porcine somatotropin have been produced using recombinant DNA technology, including somalapor, C₉₇₇H₁₅₂₇N₂₆₅O₂₈₇S₇; somenopor, C₉₃₈H₁₄₆₉N₂₅₅O₂₇₅S₇; sometripor, C₉₇₉H₁₅₂₇N₂₆₅O₂₈₇S₈; and somfasepor, C₉₃₈H₁₄₆₅N₂₅₇O₂₇₈S₆.

Somavubove is somatotropin (ox), 127-L-Leu-, which is one of the four naturally occurring molecular variants in bovine pituitary somatotropin, and which is used as a galactopoietic agent.

Somenopor is somatotropin (pig clone pPGH-1 reduced), N-L-Ala-32-de-L-Glu-33-de-L-Arg-34-de-L-Ala-35-de-L-Tyr-36-de-L-Ile-37-de-L-Pro-38-de-L-Glu- comprising 190 amino acids, which is used as a porcine growth hormone.

Sometribove is somatotropin (ox), 1-L-Met-127-L-Leu- comprising 191 amino acids, which is used as a veterinary growth stimulant.

Sometripor is a recombinant porcine somatotropin, C₉₇₉H₁₅₂₇N₂₆₅O₂₈₇S₈.

Somfasepor is a recombinant porcine somatotropin, C₉₃₈H₁₄₆₅N₂₅₇O₂₇₈S₆.

Somidobove is recombinant bovine somatotropin, C₁₀₂₀H₁₅₉₆N₂₇₄O₃₀₂S₉, which is used as a veterinary growth stimulant.

Teprotide is bradykinin potentiator B, 2-L-Trp-3-de-L-Leu-4-de-L-Pro-8-L-Glutamine- comprising 9 amino acids, which is used as an angiotensin-converting enzyme inhibitor.

Teriparatide is a protein comprising 34 amino acids which is used as a bone resorption inhibitor and an osteoporosis therapy adjunct.

Thymalfasin is thymosin α1 (ox) comprising 28 amino acids, which is used as an antineoplastic agent, in treating hepatitis and infectious diseases, and as a vaccine enhancer.

Thymopentin is a pentapeptide used as an immunoregulator.

Triptorelin is luteinizing hormone-releasing factor (pig), 6-D-Trp comprising 10 amino acids, which is used as an antineoplastic agent.

Vapreotide comprises 8 amino acids having one disulfide bridge, which is used as an antineoplastic agent.

Vasopressin in the 8-L-Arg- or 8-L-Lys- form comprises 9 amino acids having one disulfide bridge, which is used as an antidiuretic hormone.

The continuing expansion of the biotechnology industry and the use of biotechnology research tools and methods is bringing even more and different types of protein-based therapeutic agents into clinical trials and eventually the marketplace. Consider, for example, the following protein agents: myoglobin; hemoglobin; β-lactoglobulin; immunoglobulin-G (IgG); antihemophilic factor (Factor VIII); lysozyme; ubiquitin; platelet-activating factor (PAF); tumor necrosis factor-α (TNF-α); tumor necrosis factor-β (TNF-β); macrophage inflammatory protein (MIP); heparin; and eosinophil cationic protein (ECP). Further, protein-based drugs which have recently been approved include a platelet growth factor, a recombinant factor IX, a monoclonal antibody for non-Hodgkin's B-cell lymphoma, an improved interferon alpha for treatment of hepatitis C, and a fibroblast-derived artificial skin for treating wounds and burns.

Having above-described in detail the makeup of the starting material reactants used in the preparation processes of the present invention, including the above-described condensation process involved therein, there will now be described in the paragraphs which follow the details of the preparation process of the present invention itself.

The preparation processes of the present invention provide novel improved Schiff base condensation adduct final products as defined herein. Said processes comprise first producing a reaction mixture comprising the protein and aromatic o-hydroxy aldehyde starting material reactants. This reaction mixture is prepared by bringing the protein component reactant and the aromatic o-hydroxy aldehyde component reactant into intimate contact with each other in an aqueous environment.

The expressions “starting material reactant”, “component reactant”, and “reactant” are used herein to refer to the protein and aromatic o-hydroxy aldehyde entities which react to form a Schiff base condensation adduct.

The expression “in an aqueous environment” indicates that the solvent for the reaction mixture is water and that this is the medium in which the reaction takes place. The water of condensation which is formed during the reaction therefore also becomes an indistinguishable part of this “aqueous environment”.

After the starting material reactants are brought together to form the reaction mixture, the preparation process of the present invention immediately proceeds with the Schiff base condensation reaction. The expression “Schiff base condensation reaction” is used herein to refer to the reaction which is well known to the skilled person in the art of organic chemistry and the synthesis of organic chemical compounds. The basic Schiff base condensation reaction may be schematically represented as follows:

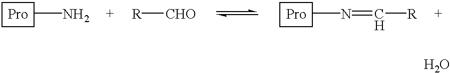

wherein

[symbol] =Protein shown in fragmentary form, since at least one amino acid thereof has a primary amine group, which is shown as attached to the Protein fragment. It is important to note that the formation of a Schiff base adduct is an equilibrium reaction which may also result in the separation of the adduct into its constituent components, and that the rate of this decomposition reaction may be as rapid as the basic reaction which initially leads to the formation of the condensation adduct.

The condensation process of the preparation process of the present invention is driven substantially to completion. The expression “substantially to completion” as used herein is intended to mean that the reaction is one which is quantitative, i.e., proceeding wholly or almost to completion. The condensation reaction of the preparation process of the present invention is made quantitative by removing from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

The term “preparation process” used herein in connection with the process embodiment of the present invention is intended to encompass the Schiff base condensation reaction, as well as the process steps which are taken in order to drive said reaction to completion. This latter portion of the overall process is accomplished by removing substantially all of the water from the aqueous environment of the reaction mixture.

For the preparation process described herein, the conditions which are effective to drive said condensation reaction substantially to completion comprise those which change the water present from the liquid phase to the gaseous or solid phase whereby it is removed from the immediate environment of said condensation reaction. In the process of the present invention said conditions must also be characterized by scalability, i.e., the ability to be readily and efficiently adapted to large, manufacturing scale production, and by reproducibility, i.e., the ability to be carried out successively without substantial deviation in end result. Since the reaction mixture in its aqueous solvent is established at ambient temperatures or elevated temperatures below the boiling point of water, the water present will, naturally, be in the liquid phase. In order to drive this reaction to completion, the water must be totally and quickly removed from the immediate vicinity of the reaction mixture. This cannot be done simply by separating the condensation adduct final product from the aqueous solvent and discarding the latter. The fact that the reaction is in equilibrium and that no precipitated product is formed precludes this approach.

There are two approaches taken in the preparation processes of the present invention. In one approach, the water is turned to a vapor or gas and removed, e.g., by spray-drying, and this embodiment of the present invention is referred to as taking place at temperatures above 0° C. In the other approach, the water is turned to a solid and removed, e.g., by lyophilization, and this embodiment of the present invention is referred to as taking place at temperatures of ₀° C. or below.

The first approach taken in the preparation process of the present invention is to remove the water by changing it from the liquid phase to the gaseous or vapor phase. Such a step is usually accomplished with a fair amount of rapidity. Where the water is changed from the liquid phase to the gaseous or vapor phase, water evaporation is involved. Consequently, it will be necessary to input energy into the process in order to satisfy the latent heat of evaporation (LHE) of the water, which is the amount of heat energy absorbed by a unit weight of the water as it passes from the liquid to the vapor state. The amount of energy required may be calculated from the equation: LHE=0.02T²/E, where T is the thermodynamic scale boiling point of the water and E is the molecular elevation of the boiling point of a solution. A related value is the specific latent heat of vaporization (SLHV), which is the number of joules required to convert 1 gram of substance from liquid into vapor without a change in temperature. For water at 100° C. this value is 2257 J.

The conditions under which the preparation process of the present invention is carried out are those which optimize the energy input to the process necessary most efficiently to separate the water of the aqueous environment in which the condensation reaction takes place, including water produced by said condensation reaction itself, from the starting material reactants and the condensation adduct final product.

The expression “energy input to the process” used herein is intended to refer to all forms of energy, individually and collectively, and to their employment in the preparation process of the present invention whereby the water of the aqueous environment is changed from the liquid phase to the vapor or solid phase. Included therein is first the atomic heat, which is the amount of heat energy necessary to raise one gram of the water from 0° to 1° C. A related concept is molecular heat, which is the amount of heat energy necessary to raise one mole of the water by 1°, i.e., specific heat×molecular weight. This input of heat energy will raise the reaction mixture and its aqueous environment to the desired temperature.

The next input of heat energy is that necessary to satisfy the heat of evaporation, which in the above discussion has been described in detail. Thereafter, mechanical energy must be applied to the reaction mixture in its aqueous environment in order to carry out the spray-drying step in which the water is completely evaporated. Thus, at temperatures above 0° C. the conditions which optimize energy input to the process comprise

(a) heating said reaction mixture in said aqueous environment to the highest temperature consistent with maintaining the integrity of the protein starting material reactant and the condensation adduct final product, as well as consistent with optimal efficiencies and economies for carrying out said preparation process including said condensation reaction.

The expression “integrity of the protein starting material reactant and the condensation adduct final product” as used herein in association with the upper limit of temperatures which may be employed, is intended to mean essentially that the protein component of the starting materials and/or products is not subject to any significant degradation as the result of such heating, i.e., denaturing which would produce any loss in biological activity, or which would interfere with release, especially sustained release of the final product from its site of administration, e.g., as a subcutaneous or parenteral depot of solid pellets.

The expression “optimal efficiencies and economies” made with reference to carrying out the preparation process of the present invention, including the condensation reaction, is intended to mean that due consideration must be given, when choosing the temperature of the reaction mixture and numerous other process parameters described herein, to carrying out said process with a view toward obtaining the most efficient process possible, as well as the process which affords the final product at the lowest cost consistent with the other choices. Thus, choices of process parameters which provide the highest yield of final product are only adhered to if the other efficiencies of the process are commensurate in quality, and only if the resulting process is one the economies of which are consistent with the best obtainable. It is well within the skill of the artisan to balance these requirements so as to achieve the best all around process.

Accordingly, taking into account the impact of all of the above considerations, typically said reaction mixture is heated to a temperature of from 25° C. to 125° C., preferably from 40° C. to 120° C., more preferably from 50° C. to 115° C., more preferably still from 60° C. to 110° C., and most preferably from 75° C. to 105° C., while maintaining the aqueous environment in the liquid phase by the application of elevated pressure where necessary. Achieving temperatures above the ambient boiling point of water, i.e., 100° C. while the aqueous environment is still in the liquid phase, can be accomplished through the use of elevated pressures.

The next step in the process of preparation wherein the temperature is above 0° C. comprises:

(b) subdividing said reaction mixture in said aqueous environment into the smallest droplets consistent with maintaining the integrity of the protein starting material reactant and the condensation adduct final product, as well as consistent with optimal efficiencies and economies for carrying out said preparation process including said condensation reaction.

In the context of the above step, maintaining the integrity of the protein will depend to some extent on the size of that protein. Thus, very large proteins may modify upwards the average diameters of droplets which are useful in this step. Typically, however, the reaction mixture in said aqueous environment is divided into droplets having an average diameter of from 1.0 μm to 5.0 mm, preferably from 10 μm to 1.0 mm, more preferably from 100 μm to 900 μm, more preferably still from 200 μm to 800 μm, and most preferably from 300 μm to 700 μm.

The preparation process of the present invention includes an embodiment wherein said starting material reactants are brought into intimate contact with each other in droplet form, i.e., formation of said condensation adduct final product occurs immediately prior to or substantially simultaneously with dispersion of said final product in droplet form. Intimate admixture of said starting material reactants in droplet form is achieved by mechanical action sufficient to bring said starting material reactants into contact with each other while at the same time not mechanically degrading the protein component of said condensation adduct. The choice of a particular mechanical mixing device will be within the skill of the artisan supplied with the description herein of the various parameters which must be considered.

One of the most important factors is the nature of the protein component involved and the amount of shear stress which it can sustain in aqueous solution before experiencing degradation of its structural integrity. This can be readily determined using routine tests of structural integrity such as electrophoresis to measure the effect, if any, which the mechanical mixing device selected has on said integrity of the protein to be used. Such routine testing may be necessary since the resistance of a given protein to mechanical shear stress in aqueous solution is by and large not totally predictable due to the capacity of larger peptides to undergo multiple folding which can confer elements of structural stability. On the other hand, problems can be avoided from the outset by choosing a mechanical mixing device which has a gentle action. Selecting the device with a view toward avoiding significant levels of shear stress in solution will frequently avoid the need for any of the above-mentioned testing.

A number of suitable mechanical mixing devices would readily suggest themselves to the artisan. For example, the mixing vessel may be stationary and utilize the rotational or other type of motion of elements such as rods, paddles or other types of stirrers to achieve mixing through gentle agitation. Where it is desired to carry out the condensation process on a continuous basis, the mixing apparatus may take the form of a trough in which the starting material reactants enter at one end and the reaction mixture and condensation adduct final product are discharged at the other end. Agitation in such an apparatus may be achieved using a slow moving worm which works in the solution and lifts final product off of the heating surface to distribute it through the solution and slowly convey it through the trough. Rocking of the entire trough can also be used in combination with baffles which increase the residence time of the solution in the trough. Both of these types of mixing devices are characterized by low heat transfer coefficients, and a more rapid heat exchange may be achieved by using a double-pipe arrangement in which the reaction mixture is carried in the central pipe with the countercurrent flow of the heating medium in the annulus between the pipes. Agitation in this type of apparatus is often achieved by the use of a shaft which rotates in the central pipe and carries blades which scrape the heat transfer surface, permitting high heat transfer coefficients to be obtained.

Mixing devices can be more passive in design and not utilize heat transfer, such as a stirred reaction vessel. For larger production levels, calandria may be employed for heating and the downcomer, which must be large enough to accommodate the flow of the reaction mixture, commonly houses an impeller, with forced circulation increasing the heat transfer to the reaction mixture. A continuous process in which close control of the final product is important may be carried out using mixing devices which concentrate the reaction mixture. In a vacuum reactor vessel, typically hot concentrated reaction mixture would be fed to an agitated reacton chamber maintained at low pressure. The reaction mixture can be permitted to boil and cool adiabatically to the boiling point corresponding to the operating pressure of the vessel.

Another type of mixing device which would be suitable for use in the process of the present invention utilizes streams of the reaction mixture produced by, e.g., hydraulic pumps which induce sufficient turbulence in said streams to assure intimate admixture of the components. The selected mechanical action may also take the form of separate sprays of each starting material reactant directed in such manner with respect to each other that maximum commingling, collision, and contact of said droplets is achieved. Spraying apparatus may be used in this process which comprise simple mechanical or hydraulic pumping means sufficient to impart the energy necessary to divide an aqueous stream containing said starting material reactants into droplets within the size ranges above described, which are required to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

The above-described pumping means can be used in conjunction with a nozzle means whereby mechanical shearing forces are applied to streams of aqueous solutions containing the starting material reactants, as a result of which said streams are divided into successively smaller droplet total volumes until the desired droplet size is achieved.

There may also be used in the preparation process of the present invention spraying apparatus comprising gas stream generators and means for dispersing said aqueous stream of said starting material reactants therein so as to be entrained thereby in droplet form having the desired droplet size. In particular, said gas is substantially inert with respect to said starting material reactants and said condensation adduct final product. Said gas consists of air, nitrogen, or helium, among others, which has been compressed to a pressure sufficiently high to provide a gas stream having the volume and velocity required to entrain said droplets of said starting material reactants and assure a commingling, contact and collision thereof sufficient to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

The spraying apparatus suitable for use in the method of the present invention comprises any suitable combination of the above-described gas stream generators and associated dispersing means together with said above-described mechanical or hydraulic pumping means and associated nozzle means. Where the temperature of the aqueous environment including the reaction mixture is to be maintained above the normal boiling point of water, i.e., 100° C., this may be accomplished by maintaining the system under elevated pressures, which will elevate the boiling point of water in the system in a predictable manner. It will also be understood that once the reaction mixture and aqueous system have been emitted as fine droplets by the spraying apparatus, that there will be an immediate and significant drop in the temperature of said droplets. For example, it is possible to maintain a temperature of 115° C. for the reaction mixture and aqueous environment in the inlet portion of the spraying apparatus through the use of elevated pressure, and once the reaction mixture and aqueous system have left the nozzle means of the spraying apparatus, their temperature will be observed to have dropped to 80° C.

In still another embodiment of the present invention the intimate admixture of said starting material reactants in droplet form is achieved by mechanical action in the form of a rotating disc over the surface of which an aqueous stream comprising each said reactant starting material is directed. A separate disc for each starting material reactant may be utilized, or else a single disc may be used which is fashioned to accommodate both said starting material reactant aqueous streams. Each said aqueous stream traverses said disc in such manner that it is propelled from the edge of said disc in droplet form; and the speed of said rotating disc is varied so as to impart sufficient energy to divide each said aqueous stream into droplets of such size and speed that maximum commingling, collision, and contact of said droplets is achieved.

The commingling of said starting material reactants takes place under conditions which have been adjusted with regard to temperature, humidity and pressure so as eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants. The temperature, for example, will generally fall within the ranges above-described wherein typically said reaction mixture is heated to a temperature of from 25° C. to 125° C., preferably from 40° C. to 120° C., more preferably from 50° C. to 115° C., more preferably still from 60° C. to 110° C., and most preferably from 75° C. to 105° C., while maintaining the aqueous environment in the liquid phase by the application of reduced pressure where necessary.

The spinning disc may be housed in an apparatus in which it is possible to maintain reduced pressures by using, e.g., vacuum pumping means, although this is not a typical arrangement. Such elevated pressures may be used to increase the boiling point of the reaction mixture and aqueous system, as above-discussed. An example of such a spinning disc sprayer is the Niro mobile spray dryer available from Niro Atomizer of Denmark. This device has a chamber 600 mm in cylindrical height and 800 mm in diameter, with a conical base having a 60° angle of conicity. When operated at atmospheric pressure, the disc speed will be in the range of 35,000 to 40,000 rpm, and the flow rate of drying air will be 80 kg/hr.

The combination of the above-described heating of the reaction mixture and aqueous environment, together with the mechanical energy imparted thereto during its separation into small droplets, will sufficiently energize the water molecules therein allow them to enter the gas or vapor phase. In order to further facilitate the removal of the water from the reaction mixture and aqueous environment, it is preferred to additionally employ a stream of air to carry away the vaporized water. The input of energy from the moving stream of air directly enhances the vaporization of the water, and generally the higher the velocity of the stream of air, the higher the enhancement of vaporization. The enhancement of vaporization is further improved by the use of an air stream having elevated temperatures, e.g., from 75° to 150° C., preferably 90° to 110° C. The heated air imparts additional energy to the vaporization process. Vaporization of the water is still further enhanced by using a heated air stream which is dry, i.e., which is low in humidity, thereby improving the ability of the heated air stream to contain additional quantities of water vapor. The humidity of the heated air stream is preferably from 1% to 20% relative humidity, preferably 2% to 10% relative humidity.

Accordingly, the last step of the preparation process of the present invention is as follows:

(c) providing said droplets thus formed with the highest comparative velocity, referenced to a gas inert thereto through which they pass, which is consistent with maintaining the integrity of the protein starting material reactant and the condensation adduct final product, as well as consistent with optimal efficiencies and economies for carrying out said preparation process including said condensation reaction.

The expression “a gas inert thereto through which they pass”, referring to both the droplets of reaction mixture and aqueous environment as well as the droplets of condensation adduct final product which are formed, is intended to mean any gas which is inert with respect to said final product. Such common gases as nitrogen and helium, which are readily available and which are inert under these circumstances, may be used. As a practical matter, however, it will ordinarily be difficult to find a more suitable inert gas that ambient air. Air is also most likely to be consistent with optimal efficiencies and economies for carrying out the preparation process of the present invention.

The comparative velocity to which said droplets are subjected is from 0.1 m/sec to 5.0 m/sec, preferably from 0.2 m/sec to 4.0 m/sec, more preferably from 0.3 m/sec to 3.0 m/sec, more preferably still from 0.4 m/sec to 2.0 m/sec, and most preferably from 0.5 m/sec to 1 0 m/sec. This velocity takes into account the relative velocity of the stream of inert gas, which may flow with, against, across, or at any angle to, the stream of said droplets.

By following the above procedures, it is possible to divide the reaction mixture in said aqueous environment into droplets having an average diameter of from 1.0 μm to 5.0 mm, preferably from 10 μm to 1.0 mm, more preferably from 100 μm to 900 μm, more preferably still from 200 μm to 800 μm, and most preferably from 300 μm to 700 μm. It will be understood that the smaller the droplet, the more efficient will be the vaporization and removal of the water from the reaction mixture and aqueous environment. This is due primarily to the greatly expanded surface area available to the water molecules from which they may move from the liquid phase to the vapor phase and be carried away by the surrounding stream of inert gas.

It is further possible to carry out these condensation processes under conditions of reduced moisture in order to accelerate the rate of water removal. This will assist in driving the condensation reaction to completion, and consistent therewith the amount of moisture present in the condensation adduct final product will be from 3.0% to 0.001% by weight based on the weight of the final product, preferably from 2.0% to 3.0% by weight, based on the weight of said final product. However, it is also possible to further remove additional amounts of moisture in order to provide a drier final product which resists caking and has improved stability and other handling characteristics. The amount of moisture present in the condensation adduct final product may thus be as low as from 0.1% to 0.001% by weight, or from 0.05% to 0.005% by weight, or even as low as from 0.03% to 0.01% by weight, based on the weight of the final product. It must be cautioned, on the other hand, that it may be necessary to have substantially higher amounts of moisture present in the final product, since many proteins exhibit instability if they are totally dehydrated. Consistent with the object of maintaining the integrity of the final product, the amounts of moisture present in the final product may be in the range of from 3.0% to 20.0% by weight, preferably from 5.0% to 15.0% by weight, and more preferably from 8.0% to 12.0% by weight, based on the weight of the final product.

As mentioned further above, there are two approaches taken in the preparation processes of the present invention. In the first approach described above, the water is turned to a vapor or gas and removed, e.g., by spray-drying, and this embodiment of the present invention is referred to as taking place at temperatures above 0° C. In the second approach, described in the paragraphs which follow, the water is turned to a solid and removed, e.g., by lyophilization, and this embodiment of the present invention is referred to as taking place at temperatures of 0° C. or below.

The second approach taken in the preparation process of the present invention is to remove the water by changing it from the liquid phase to the solid phase. Such a step is not usually accomplished with rapidity, as is the step of conversion to the vapor phase. Where the water is changed from the liquid phase to the solid phase, freezing of water is involved, which essentially requires the removal of energy from the aqueous environment of the reaction mixture. However, in order to remove energy from said aqueous environment, i.e., to lower its temperature and ultimately change it into the solid phase, it will be necessary to employ energy in the preparation process of the present invention. For example, this would involve the use of a refrigerating or rapid heat exchange system and bringing it into contact with the aqueous environment. Consequently, it will be necessary to input energy into the preparation process in order to remove sufficient heat energy for a given unit weight of the water involved, to reduce its temperature and ultimately change it to the solid phase.

One method of accomplishing the above-described removal of heat energy from said aqueous environment of the preparation process is by freeze-drying, or lyophilization of said aqueous environment, including the reaction mixture. In accordance with the present invention, such a freeze-drying process would be carried out in such manner that said reaction mixture is cooled to a temperature of from −110° C. to 0° C., preferably from −45° C. to −5° C., more preferably from −40° C. to −10° C., more preferably still from −35° C. to −15° C., and most preferably from −30° C. to −20° C., while maintaining the aqueous environment in the solid phase, i.e., frozen. This drying process is essentially one in which the aqueous solvent is removed by first freezing it and then removing it by sublimation in a vacuum environment.

The reduced pressure to which the cooled reaction mixture in the aqueous environment is subjected in order to increase the rate of water removal is from 5.0 mmHg absolute to 0.0001 mmHg absolute, preferably from 1.0 mmHg absolute to 0.0005 mmHg absolute, more preferably from 0.5 mmHg absolute to 0.001 mmHg absolute, more preferably still from 0.2 mmHg absolute to 0.005 mmHg absolute, and most preferably from 0.1 mmHg absolute to 0.01 mmHg absolute. Such reduced pressures can be obtained using vacuum pumps of various capacities and known construction.

In a conventional manner of carrying out a freeze-drying process of the type contemplated herein, the reaction mixture aqueous solution is filled into suitable containers such as vials which are then placed in a temperature-controlled environment such as a large drying chamber. The condensation adduct final products involved will eventually be employed in the treatment of human and animal diseases and conditions. Accordingly, it is efficient to process such products in a collection of small batches such as vials, since these individually provide an appropriate volume to surface ratio for carrying out the freeze-drying process and a large number of vials can be processed at one time.

The temperature in the drying chamber is then brought to and maintained at a level of about −40° C., whereafter the reaction mixture aqueous solution quickly becomes a solid consisting of ice and solid solute, i.e., condensation adduct final product. The ice crystallizes and the solute either crystallizes or becomes a glassy solute, depending on the final product involved and the nature of the freeze-drying process being carried out. The drying chamber is then evacuated by means of vacuum pumps, and the temperature in the drying chamber is increased to initiate sublimation of the ice to vapor stage of the freeze-drying, often referred to as the primary drying step. The water vapor which is produced by sublimation is transported through the partially dried condensation adduct on its way to a condenser chamber equipped with surfaces maintained at even lower temperatures of about −60° C., where the vapor is condensed and thereby removed. With increasing temperature of the condensation adduct product, the rate of primary drying increases, but caution must be exercised not to exceed the maximum temperature for maintaining the integrity of the product.

The primary drying step removes all of the ice in the initial condensation adduct product. However, the amount of moisture in the product, which is contained in a dissolved state in the amorphous portions of the product, is still substantial, on the order of about 20% to 50% by weight, depending upon the makeup of said product. The removal of this remaining water is accomplished during the secondary drying stage, which is typically carried out at elevated product temperatures. These temperatures, however, are not as high as those employed in the spray-drying processes of the present invention described herein. Normally, it is preferred to utilize the freeze-drying processes rather than the spray-drying processes of the present invention, since the former, being low-temperature, are more likely to be free of any destructive effects on the protein-containing final products. Freeze-drying processes also have the advantage of making the prevention of microbe and particulate contamination more readily obtainable. On the other hand, freeze-drying processes suffer from the disadvantage of involving higher capital installation costs and higher energy consumption costs for manufacturing than spray-drying processes. For either type of process, however, the proteinaceous nature of the final product creates substantial challenges to maintaining conformational stability in the final product.

The above-mentioned amorphous phase of the condensation adduct comprises uncrystallized product solute and uncrystallized water. As a practical matter, the water does not crystallize into ice at the equilibrium point, but must be supercooled 10-15° below that point before it will nucleate and crystallize. The amount of supercooling required is dictated by the solute makeup and temperature and residence time in the drying chamber, as well as by the size and material makeup of the container vial and by the presence of any particulate matter in the condensation adduct aqueous solution which can provide heterogeneous nucleation sites for ice formation. Scale-up problems in this regard can be generated by the sterile, particulate-free environments of manufacturing sites associated with the production of therapeutic agents for animals and humans, of which the condensation adduct products are most likely to consist. Such environments limit the chance of particulate nucleation sites, resulting in the need for a greater degree of supercooling of the adduct product, which in turn controls the size of the ice crystals formed. Ice crystal size is important because it controls the size of the pores or channels created in the ice crystals during sublimation, which affects the surface area of these pores available during the sublimation process.

Ultimately, the rate of sublimation as well as the rate of secondary drying are significantly affected by these factors. A 10° C. increase in supercooling can lead to an order of magnitude increase in primary drying time. The degree of supercooling should be limited to 10°-15° C. and should be uniform throughout the drying chamber and from vial to vial.

The drying chamber temperature and residence time parameters selected to give optimal results where the objective is to obtain a uniform degree of supercooling and freezing behavior consist of first cooling all of the condensation adduct product to a temperature below 0° C., but above the temperature that causes nucleation and crystallization, about −5° to −10° C. Subsequently, the drying chamber temperature is lowered to a moderate level to induce ice crystallization in all of the container vials, about −20° to −30° C. After this has taken place, the drying chamber temperature is lowered well below the lowest eutectic temperature where the solute is crystalline or below the glass-transition temperature where the solute is amorphous, about −40° C. Once the eutectic system has crystallized it is completely solid and primary drying can then proceed.

Where the solute system tends to remain amorphous a tempering or annealing process may be employed in which the condensation adduct product temperature is increased at least several degrees above the glass-transition temperature for several hours in order to allow crystallization of the solute, after which the temperature in the drying chamber is again lowered before primary drying is begun. It must also be noted that during this process ice formation leads to a concentration of all solutes, which would include dissolved salts including, e.g., where the condensation adduct product is dissolved in a mildly saline solution. The result would be an increasing concentration of NaCl which ultimately might lead to degradation of said product.

The primary drying stage is carried out at the maximum allowable temperature rather than the highest temperature possible in order to prevent product degradation. This temperature will be the eutectic temperature where the solute is crystalline and the collapse temperature, or eutectic melt temperature where the solute is amorphous. Drying above the maximum allowable temperature results in an unacceptable product which lacks definite geometry. The maximum allowable temperature, which is readily determinable by thermal-analysis methods, electrical resistance measurements or microscopic analysis of product vs. temperature, can vary over a significant range and must be determined as the first step in establishing the freeze-drying process parameters.

The above-described preparation processes may also be carried out under conditions of reduced moisture whereby the rate of water removal is accelerated and the overall amount removed is increased. This is consistent with the goal of driving the condensation reaction to completion by eliminating from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

Consistent with that goal, the amount of moisture present in the condensation adduct final product will correspondingly be from 3.0% to 0.001% by weight based on the weight of the final product, preferably from 2.0% to 3.0% by weight, based on the weight of said final product. After the condensation reaction is complete, however, it is also possible to further remove additional amounts of moisture from the final product where that is desired in order to prevent caking, enhance stability, improve handling or for other purposes apparent to the artisan. Accordingly, the amount of moisture present in the condensation adduct final product may be as low as from 0.1% to 0.001% by weight, or from 0.05% to 0.005% by weight, or even as low as from 0.03% to 0.01% by weight, based on the weight of the final product.

However, depending upon the nature of the condensation adduct final product, especially the protein component thereof, it may be necessary to have substantially higher amounts of moisture present in the final product, since many proteins are unstable if all of the water is removed from them. Thus, consistent with maintaining the integrity of the final product, it may be desirable to have amounts of moisture present in the final product in the range of from 3.0% to 20.0% by weight, preferably from 5.0% to 15.0% by weight, and more preferably from 8.0% to 12.0% by weight, based on the weight of the final product.

### DESCRIPTION OF PREFERRED EMBODIMENTS

The following examples are presented in order to further illustrate the novel processes and products of the present invention, but are not intended to in any way be taken as limiting the present invention.

### EXAMPLE 1

Condensation Adducts of Met-pST and Other Aldehydes Prepared by Lyophilization

A 2.50 mM solution of o-vanillin was prepared by dissolving 76.1 mg of o-vanillin in 200 ml of distilled water. Minimal heating and sonication were needed to fully dissolve the aldehyde. Aqueous solutions of vanillin, salicylaldehyde, and benzaldehyde (all 2.50 mM) were prepared in a similar manner. Dry, lyophilized met-pST (21.9 mg, 21858 g/m, 1.00 μmole) was dissolved in 2.00 ml of each aldehyde solution at room temperature. The pH was adjusted to 8.0 with dilute sodium hydroxide solution (dilute acetic acid was used if the pH needed to be adjusted down). The final solutions contained 1.00 μmole of protein and 5.00 μmole of aldehyde. The solutions were allowed to sit at room temperature for an hour, and were then placed in 20 mL lyophilization flasks and frozen at −28° C. in a freezer for 16 hours. The frozen samples were then placed on a manifold type freeze dryer and the flasks evacuated. The pressure was held at <1.0 mm for 24 hours. The flasks were then brought back up to atmospheric pressure, and the weight of the materials recovered was determined. In each case the weight determined was within experimental error with reference to the combined weight of the aldehyde and protein starting materials. Any loss of aldehyde could not be determined at this reaction scale. Reverse phase HPLC analysis showed only peaks for met-pST monomer and the appropriate aldehyde, with >95% recovery of monomer. Electrospray mass spectral analysis was also run on each sample. The electrospray samples were dissolved in a solution of 0.1% trifluoroacetic in 2-methoxyethanol (˜0.1 mg/ml) with the aid of sonication. The samples were made up less than 5 minutes prior to analysis, as control experiments indicated that partial hydrolysis of the material occurred if the solution was allowed to sit for longer periods of time.

The above-described preparations represent both Schiff base condensation adduct final products falling within the scope of the present invention, as well as such adducts which are not within the scope of the present invention because they were prepared using other than an aromatic o-hydroxy aldehyde. Table 1-A below summarizes the preparation of each test sample. Table 1-B below presents an analysis of each of said samples, including an indication of the predicted number of equivalents of aldehyde to protein for complete Schiff base adduct formation. The percent yield for each sample is determined on the basis above-described. The percent yield by weight for the freeze-drying process is always quantitative within experimental error for all aldehyde and protein starting materials, since the only loss of material is from sublimed aldehyde, and this only in the case where non-o-hydroxy aldehydes are used, and even in that case the loss is too small to measure. The percent yield of Schiff base is equivalent to the mass of the final product, which is always 100% of theoretical for the reasons just mentioned, ×the conversion yield. The conversion yield, in turn, is obtained by taking the actual average number of equivalents of aldehyde as determined by Electrospray Mass Spectrophotometry and dividing it by the number of equivalents predicted on a theoretical basis, and multiplying the result by 100 in order to express the conversion yield as a percentage. The values thereby obtained are a further indication of the efficiency of conversion to Schiff base obtained using the preparation processes of the present invention.

Finally, the pH of each sample was determined prior to carrying out the test procedure involved in order to demonstrate the importance of maintaining the pH at 7.0 or higher for obtaining high yields.

TABLE 1-A

| PROD. NO. | METHOD | PROTEIN | MOL. WT. | BINDING SITES | ALDEHYDE |
|---|---|---|---|---|---|
| 1a | Freeze Dry −28° C. | Myoglobin | 16951 | 20 | o-vanillin |
| 1b | Freeze Dry −28° C. | Myoglobin | 16951 | 20 | vanillin |
| 1c | Freeze Dry −28° C. | Myoglobin | 16951 | 20 | salicylaldehyde |
| 1d | Freeze Dry −28° C. | Myoglobin | 16951 | 20 | benzaldehyde |
| 1e | Freeze Dry −28° C. | β-Lactoglobulin | 18365 | 16 | o-vanillin |
| 1f | Freeze Dry −28° C. | β-Lactoglobulin | 18365 | 16 | vanillin |
| 1g | Freeze Dry −28° C. | β-Lactoglobulin | 18365 | 16 | salicylaldehyde |
| 1h | Freeze Dry −28° C. | β-Lactoglobulin | 18365 | 16 | benzaldehyde |
| 1i | Freeze Dry −78° C. | Met-pST | 21858 | 12 | o-vanillin |
| 1j | Freeze Dry −78° C. | Met-pST | 21858 | 12 | vanillin |
| 1k | Freeze Dry −78° C. | Met-pST | 21858 | 12 | salicylaldehyde |
| 1l | Freeze Dry −78° C. | Met-pST | 21858 | 12 | benzaldehyde |
| 1m | Freeze Dry −28° C. | Met-bST | 21875 | 12 | o-vanillin |
| 1n | Freeze Dry −28° C. | Met-bST | 21875 | 12 | vanillin |
| 1o | Freeze Dry −28° C. | Met-bST | 21875 | 12 | salicylaldehyde |
| 1p | Freeze Dry −28° C. | Met-bST | 21875 | 12 | benzaldehyde |
| 1q | Freeze Dry −28° C. | Lysozyme | 14306 | 7 | o-vanillin |
| 1r | Freeze Dry −28° C. | Lysozyme | 14306 | 7 | vanillin |
| 1s | Freeze Dry −28° C. | Lysozyme | 14306 | 7 | salicylaldehyde |
| 1t | Freeze Dry −28° C. | Lysozyme | 14306 | 7 | benzaldehyde |
| 1u | Freeze Dry −28° C. | Met-bST | 21875 | 12 | o-vanillin |
| 1v | Freeze Dry −28° C. | Met-bST | 21875 | 12 | vanillin |
| 1w | Freeze Dry −28° C. | Met-bST | 21875 | 12 | salicylaldehyde |
| 1x | Freeze Dry −28° C. | Met-bST | 21875 | 12 | benzaldehyde |
| 1y | Freeze Dry −28° C. | Lysozyme | 14306 | 7 | o-vanillin |
| 1z | Freeze Dry −28° C. | Lysozyme | 14306 | 7 | o-vanillin |
| 1aa | Freeze Dry −28° C. | Lysozyme | 14306 | 7 | o-vanillin |
| 1bb | Freeze Dry −28° C. | Lysozyme | 14306 | 7 | o-vanillin |
| 1cc | Freeze Dry −28° C. | Lysozyme | 14306 | 7 | o-vanillin |
| 1dd | Freeze Dry −28° C. | Lysozyme | 14306 | 7 | o-vanillin |
| 1ee | Freeze Dry −28° C. | Met-pST | 21858 | 12 | o-vanillin |
| 1ff | Freeze Dry −28° C. | Met-pST | 21858 | 12 | vanillin |
| 1gg | Freeze Dry −28° C. | Met-pST | 21858 | 12 | salicylaldehyde |
| 1hh | Freeze Dry −28° C. | Met-pST | 21858 | 12 | benzaldehyde |

TABLE 1-B

| PROD. | | EQUIV. | ACTUAL | % | |
|---|---|---|---|---|---|
| NO. | o-HYDROXY? | ALD./PROT. | AVERAGE | YIELD | pH |
| 1a | yes | 6 | 6.3 | 105 | 7.61 |
| 1b | no | 6 | 4.4 | 73* | 7.61 |
| 1c | yes | 6 | 5.6 | 93 | 7.54 |
| 1d | no | 6 | 1.6 | 27 | 7.71 |
| 1e | yes | 6 | 5.7 | 95 | 7.41 |
| 1f | no | 6 | 3.6 | 60 | 7.51 |
| 1g | yes | 6 | 5 | 83** | 7.59 |
| 1h | no | 6 | 2 | 33 | 7.62 |
| 1i | yes | 2 | 2 | 100 | 7.5 |
| 1j | no | 2 | 1.4 | 70 | 7.5 |
| 1k | yes | 2 | 1.8 | 90 | 7.5 |
| 1l | no | 2 | 0.6 | 30 | 7.5 |
| 1m | yes | 5 | 4.2 | 84** | 8.6 |
| 1n | no | 5 | 1.8 | 36 | 8.79 |
| 1o | yes | 5 | 4.3 | 86** | 8.66 |
| 1p | no | 5 | 3.4 | 68 | 8.86 |
| 1q | yes | 3 | 3 | 100 | 7.52 |
| 1r | no | 3 | 1.3 | 43 | 7.53 |
| 1s | yes | 3 | 2.7 | 90 | 7.67 |
| 1t | no | 3 | 0.8 | 27 | 7.5 |
| 1u | yes | 5 | 4.5 | 90 | 9.03 |
| 1v | no | 5 | 3.4 | 68 | 9.15 |
| 1w | yes | 5 | 4.7 | 94 | 8.98 |
| 1x | no | 5 | 2.8 | 56 | 9.01 |
| 1y | yes | 3 | 1 | 33 | 3.31 |
| 1z | yes | 3 | 1.8 | 60 | 4.47 |
| 1aa | yes | 3 | 2.1 | 70 | 5.62 |
| 1bb | yes | 3 | 2.4 | 80 | 6.53 |
| 1cc | yes | 3 | 2.7 | 90 | 7.55 |
| 1dd | yes | 3 | 2.8 | 93 | 8.52 |
| 1ee | yes | 5 | 5.4 | 108 | 8 |
| 1ff | no | 5 | 1.9 | 38 | 8.03 |
| 1gg | yes | 5 | 5.5 | 110 | 8.01 |
| 1hh | no | 5 | 3.3 | 66 | 8.01 |

*This is the only example of a yield above 70% for a non-o-hydroxy aldehyde adduct.
**These are the only examples of a yield below 90% for an o-hydroxy aldehyde adduct.

The preparation processes of the present invention carried out at temperatures above 0° C. are illustrated in the following example.

### EXAMPLE 2

Condensation Adducts of Met-PST and o-vanillin Prepared by Spray-Drying

A 1.00 gm sample of dry, lyophilized met-pST (45.7 μmole) was dissolved in 100.0 ml of distilled water. To this solution was added o-vanillin (34.8 mg, 228.5 mmole, 5.00 equivalents). The o-vanillin was dissolved by stirring at 40° C. for 1 hour. The pH was then adjusted to 7.50 with 0.1 N sodium hydroxide solution. The sample was fed into a Buchi model 190 Mini Spray Dryer at a rate of 2.0 ml per minute. The aspirator was set at −25 mbar, the sample inlet temperature was 110° C., and the sample outlet temperature was 75° C. The product was collected in the cyclone collector, and analyzed by reverse phase HPLC and electrospray mass spectrometry.

The above-described preparations represent Schiff base condensation adduct final products falling within the scope of the present invention, because they were all prepared using o-vanillin, an aromatic o-hydroxy aldehyde. Similar conditions were used with other aldehydes. Table 2-A below summarizes the preparation of each test sample. Table 2-B below presents an analysis of each of the test samples, including an indication of the predicted number of equivalents of aldehyde to protein for complete Schiff base adduct formation. The percent yield by weight for the spray-drying process is always quantitative within experimental error for all aldehyde and protein starting materials, since the only loss of material is from sublimed aldehyde, and this only in the case where non-o-hydroxy aldehydes are used, and even in that case the loss is too small to measure. The percent yield of Schiff base is equivalent to the mass of the final product, which is always 100% of theoretical for the reasons just explained, ×the conversion yield. The conversion yield, in turn, is obtained by taking the actual average number of equivalents of aldehyde as determined by Electrospray Mass Spectrophotometry and dividing it by the number of equivalents predicted on a theoretical basis, and multiplying the result by 100 in order to express the conversion yield as a percentage.

The values thereby obtained are a further indication of the efficiency of conversion to Schiff base obtained using the preparation processes of the present invention. The mass yields for the above-described spray-drying process are relatively low as a direct result of the reduced scale on which said process was carried out. Significant amounts of final product end up adhered to the drying apparatus instead of being recovered. Accordingly, the above-described conversion yield to product is a more accurate basis for demonstrating the comparative superiority of the process of the present invention.

Finally, the pH of each sample was determined prior to carrying out the test procedure involved in order to demonstrate the importance of maintaining the pH at 7.0 or higher for obtaining high yields.

TABLE 2-A

| PROD. | | | MOL. | BINDING | |
|---|---|---|---|---|---|
| NO. | METHOD | PROTEIN | WT. | SITES | ALDEHYDE |
| 2a | Spray-Dry | Met-pST | 21858 | 12 | o-vanillin |
| 2b | Spray-Dry | Met-pST | 21858 | 12 | isovanillin |
| 2c | Spray-Dry | Met-pST | 21858 | 12 | pyridoxal•HCl |
| 2d | Spray-Dry | Ala-pST | 21798 | 12 | vanillin |
| 2e | Spray-Dry | Ala-pST | 21798 | 12 | 2,4-dihydroxy- |
| | | | | | benzaldehyde |

TABLE 2-B

| PROD. | | EQUIV. | ACTUAL | | |
|---|---|---|---|---|---|
| NO. | o-HYDROXY? | ALD./PROT. | AVERAGE | % YIELD | pH |
| 2a | yes | 5 | 5 | 100 | 7.5 |
| 2b | no | 5 | 3.1 | 62 | 7.5 |
| 2c | yes | 3.7 | 3.4 | 92 | 7.5 |
| 2d | no | 5 | 1.7 | 34 | 7.5 |
| 2e | yes | 5 | 4.6 | 92 | 7.5 |

In the above tables of data, the yield values (%'s) are accurate to within 5-10% of the recited number. It will be noted that for all of the preparations in which an aromatic o-hydroxy aldehyde was employed and the pH was ≧7.0, that the yield was ≧90%. The only exceptions to this observation are pointed out in the relevant above-recited table. By contrast, where aromatic non-o-hydroxy aldehydes were employed, the yields were all ≦70%, even though the pH was ≧7.0 as with the o-hydroxy aldehyde samples. The only exception to this observation is pointed out in the relevant above-recited table.

The critical importance to obtaining high yields of maintaining the pH at a value ≧7.0 is illustrated by the values above-recited in Table 1-B for samples 1y through 1dd. All of these samples employed an aromatic o-hydroxy aldehyde, so that yields ≧90% would have been expected had the pH been maintained at ≧7.0. However the pH's were established at different values over a range beginning at a low of 3.31 and progressively increasing to a high of 8.52. The yield %'s showed a corresponding progression, beginning with a low of 33% and regularly increasing to a high of 93%.

What is claimed is:

1. An improved process for preparing Schiff base condensation adduct final products whose components comprise a protein having beneficial activity in animals, and an aromatic o-hydroxy aldehyde, which comprises bringing together the above-mentioned components in an aqueous environment at a pH of 7.0 or higher to form a reaction mixture, under conditions effective to drive, said condensation reaction substantially to completion by removing from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

2. A process according to claim 1 wherein said aromatic o-hydroxy aldehyde comprises one or more compounds of the formula: 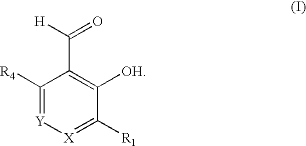
wherein:
R₁ and R₄ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂-C₆)alkenyl; —C(═O)OR₇ -—OC(═O)R₇; —S(═O)₂—S(═O)₂N(R₇)(R₉); —S(═O)₂R₇; —S(═O)₂OR₇; —C(═O)NR₇R₉; —C(═O)R₉; and —N(R₇)(R₉), where R₇ is hydrogen or (C₁-C₄)alkyl and R₉ is (C₁-C₄) alkyl;
wherein:
said alkyl, cycloalkyl and alkenyl groups defining R₁ and R₄ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁ -C₂)alkyl; sulfonyl; and sulfonamido disubstituted by (C₁ -C₂)alkyl;
X and Y are independently N, or CHR₂ or CHR₃, respectively, where R₂ and R₃ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂ -C₆)alkenyl; —C(═O)OR₁, —OC(═O)R₁₁,; —S(═O)₂; —S(═O)₂N(R₁₁)(R₁₃); and —N(R₁₁)(R₁₃), where R₁₁ is hydrogen or (C₁-C₄)alkyl and R₁₃ is (C₁-C₄)alkyl; and wherein said alkyl, cycloalkyl and alkenyl groups defining R₂ and R₃ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl.

3. A process according to claim 2 wherein said aromatic o-hydroxy aldehyde comprises o-vanillin; salicylaldehyde; 2,3-dihydroxybenzaldehyde; 2,6-dihydroxybenzaldehyde; 2-hydroxy-3-ethoxybenzaldehyde; or pyridoxal.

4. A process according to claim 1 wherein said protein having beneficial activity in animals comprises one or more members selected from the group consisting of:
proteinaceous endogenous and synthetic opioid analgesics and antagonists comprising enkephalins, endorphins, and dynorphins which are selective and nonselective agonists and antagonists of the μ, κ, and δ opioid receptor subtypes, including [Leu⁵] and [Met⁵]enkephalin; dynorphin A and B; α- and β-neoendorphin; [D-Ala², MePhe⁴,-Gly(ol)⁵]enkephalin (DAMGO); [D-Pen², D-Pen⁵]enkephalin (DPDPE); [D-Ser², Leu⁵]enkephalin-Thr⁶ (DSLET); [D-Ala², D-Leu⁵]enkephalin (DADL); D-Phe-Cys-Tyr-D-Trp-Orn-Thr-Pen-Thr-NH₂ (CTOP); [D-Ala, N-MePhe⁴, Met(O)⁵-ol]enkephalin (FK-33824); Tyr-D-Ala-Phe-Asp-Val-Val-Gly-NH₂ ([D-Ala²]deltorphin I; Tyr-D-Ala-Phe-Glu-Val-Val-Gly-NH₂ ([D-Ala², Glu⁴]deltorphin II; Tyr-Pro-Phe-Pro-NH₂ (morphiceptin); Tyr-Pro-MePhe-D-Pro-NH₂ (PL-017); and [D-Ala², Leu⁵, Cys⁶]enkephalin;
autocoids including bradykinin and kallidin produced by proteolytic reactions in response to inflammatory events selected from tissue damage, viral infections, and allergic reactions, wherein said proteins act locally to produce pain, vasodilatation, increased vascular permeability and the synthesis of prostaglandins, wherein said proteins have agonist and antagonist activity and are useful for the treatment of male infertility, for the delivery of cancer chemotherapeutic agents beyond the blood-brain barrier, and for the treatment of pain, asthma, and other chronic inflammatory diseases, including: Arg-Pro-Pro-Gly-Phe-Ser-Pro-Phe-Arg (bradykinin); Lys-Arg-Pro-Pro-Gly-Phe-Ser-Pro-Phe-Arg (kallidin); Arg-Pro-Pro-Gly-Phe-Ser-Pro-Phe (des-Arg⁹-bradykinin); Lys-Arg-Pro-Pro-Gly-Phe-Ser-Pro-Phe (des-Arg¹⁰-kallidin); Arg-Pro-Pro-Gly-Phe-Ser-Pro-Leu (des-Arg⁹-[Leu⁸]-bradykinin); Arg-Pro-Pro-Gly-Phe-Ser-[D-Phe]-Phe-Arg ([D-Phe⁷]-bradykinin); and [D-Arg]-Arg-Pro-Hyp-Gly-Thi-Ser-Tic-Oic-Arg (HOE 140), where Hyp is trans-4-hydroxy-Pro; Thi is β-(2-thienyl)-Ala; Tic is [D]-1,2,3,4-tetrahydroquinolin-3-yl-carbonyl; and Oic is (3 as, 7 as)-octahydroindol-2-yl-carbonyl; diseases and uses as adjunctive agents in the treatment of severe infections and in the management of patients who are undergoing chemotherapy or marrow transplantation, including specifically: growth factors including erythropoietin (EPO); stem cell factor (SCF); interleukins (IL-1-12); monocyte/macrophage colony-stimulating factor (M-CSF, CSF-1); P1XY321 (GM-CSF/IL-3 fusion protein); and thrombopoietin;
thrombolytic proteins useful for dissolving both pathological thrombi and fibrin deposits at sites of vascular injury, including streptokinase; tissue plasminogen activator (t-PA); and urokinase;
anterior pituitary hormones and the hypothalamic factors that regulate their use comprising: (a) somatotropic hormones including growth hormone (GH), prolactin (Prl), and placental lactogen (PL); (b) glycoprotein hormones including luteinizing hormone (LH), follicle-stimulating hormone (FSH), and thyroid-stimulating hormone (TSH); and (c) POMC-derived hormones including corticotropin (ACTH), α-melanocyte-stimulating hormone (α-MSH), β-melanocyte-stimulating hormone (β-MSH), β-lipotropin (β-LPH), and γ-lipotropin (γ-LPH); the hypothalamic factors regulating release of said hormones, including growth hormone-releasing hormone (GHRH), luteininzing hormone releasing hormone (LHRH), insulin-like growth factor (IGF-1 and IGF-2), somatostatin, and gonadotropin-releasing hormone (GnRH);
growth hormone useful as replacement therapy in growth-hormone deficient children, including: somatostatin, the synthetic analogue of somatostatin, octreotide; gonadotropic hormones including LH, FSH, and corionic gonadotropin (GC) useful in the diagnoses of reproductive disorders and in the treatment of infertility, including: urofollitropin, a human menopausal gonadotropin (hMG) from which substantially most of the LH has been removed useful for inducing ovulation, and gonadorelin, a synthetic human GnRH useful for stimulating gonadotropin secretion; synthetic GnRH agonists including: leuprolide, histrelin, nafarelin, and goserelin useful in treating endocrine disorders that are responsive to reductions in gonadal steroids;
thyrotropin (TSH), the secretion of which is controlled by thyrotropin-releasing hormone (TRH), useful for hormone replacement therapy in patients with hypothyroidism and for TSH suppression therapy in patients with nontoxic goiter or after treatment for thyroid cancer;
insulin for treating insulin-dependent diabetes mellitus patients and non-insulin-dependent diabetes mellitus patients; glucagon which has a physiological role in the regulation of glucose and ketone body metabolism, useful in treating severe hypoglycemia, and by radiologists for inhibiting the gastrointestinal tract; somatostatin, useful for blocking hormone release in endocrine-secreting tumors, including insulinomas, glucagonomas, VIPomas, carcinoid tumors, and somatotropinomas, and the synthetic analogue, octreotide;
calcitonin, a hormone acting specifically on osteoclasts to inhibit bone resorption, is useful in managing hypercalcemia and in disorders of increased skeletal remodeling, including Paget's disease; parathyroid hormone, useful in the treatment of patients with spinal osteoporosis;
aldesleukin, 125-L-serine-2-133-interleukin 2, useful as an antineoplastic agent and as an immunostimulant; alglucerase, a monomeric glycoprotein of 497 amino acids and a modified form of human placental tissue β-glucocerebrosidase, is useful as a replenisher of the glucocerebrosidase enzyme; alsactide, a synthetic corticotropin analogue. 1-β-Ala-17[L-2,6-diamino-N-(4-aminobutyl)hexanamide]-α¹⁻¹⁷-corticotropin; alteplase, a serine protease of 527 amino acids whose sequence is identical to the naturally occurring protease produced by endothelial cells in vessel walls, useful as a plasminogen activator; alvircept sudotox, a synthetic chimeric protein engineered to link the first 178 amino acids of the extracellular domain of CD₄ via two linker residues to amino acids 1-3 and 253-613 of Pseudomonas exotoxin A, useful as an antiviral agent; amlintide, a protein of 37 amino acids, useful as an antidiabetic agent; amogastrin: N-carboxy-L-Trp-L-Met-L-α-Asp-3-phenyl-L-Alaninamide; anakinra: N²-L-Met-interleukin 1 receptor antagonist useful as a nonsteroidal anti-inflammatory and as a suppressant for treating inflammatory bowel disease; anaratide acetate, atriopeptin-21 (rat), N-L-Arg-8-L-Met-21a-L-Phe-21b-L-Arg-21c-L-Tyr-, acetate, useful as an antihypertensive agent and as a diuretic; angiotensin amide, angiotensin II, 1-L-Asn-5-L-Val-, useful as a vasoconstrictor; aprotinin, a pancreatic trypsin inhibitor having 58 amino acids, useful as an enzyme inhibitor (proteinase); arfalasin, 1-succinamic acid-5-L-Val-8-(L-2-phenylglycine)angiotensin II, useful as an antihypertensive agent; argipressin tannate, vasopressin, 8-L-Arg-, tannate, useful as an antidiuretic; aspartocin, oxytocin, 4-L-Asn-, is useful as an antibiotic agent produced by Streptomyces griseus; atosiban, oxytocin, 1-(3-mercaptopropanoic acid)-2-(O-ethyl-D-Tyr)-4-L-Thr-8-L-Orn-, useful as an oxytocin antagonist; avoparcin, a glycopeptide antibiotic obtained from Streptomyces candidus; basifungin, N-[(2R,3R)-2-hydroxy-3-MeVal]-N-L-MeVal-L-Phe-N-L-MePhe-L-Pro-L-allo-Ile-N-L-MeVal-L-Leu-3-hydroxy-N-L-MeVal α₁-lactone, useful as an antifungal agent; becaplermin, recombinant human platelet-derived growth factor B, a recombinant protein produced by genetically engineered Saccharomyces cerevisiae similar in amino acid composition and biological activity to endogenous human PDGF-BB homodimer, useful for treating chronic dermal ulcers by promoting proliferation of mesenchymally-derived cells; bivalirudin, an anticoagulant, antithrombotic agent having 20 amino acids; carbetocin, 1-butyric acid-2-[3-(p-methoxyphenyl)-L-Ala]oxytocin; cargutocin, 1-butyric acid-6-(L-2-aminobutyric acid)-7-glycineoxytocin; ceruletide, 5-O-L-Pro-L-Gln-L-α-Asp-L-O-sulfo-L-Tyr-L-Thr-L-Gly-L-Trp-L-Met-L-α-Asp-L-Phe-amide, useful as a gastric secretory stimulant; cetermin, transforming human growth factor β2 having 112 amino acids; cilmostim, 1-233-colony-stimulating factor 1 (human clone p3ACSF-69 protein moiety), cyclic (7→90), (48→139), (102→146)-tris(disulfide) dimer, useful as a hematopoietic agent (macrophage colony-stimulating factor); colistimethate sodium, a colistin A component useful as an antibacterial agent; corticorelin, ovine triflutate, corticotropin-releasing factor (sheep), trifluoroacetate salt, useful as a diagnostic aid for adrenocortical insufficiency and Cushing's syndrome, and as a corticotropin-releasing hormone; cosyntropin, tetracosactide acetate, α¹⁻²⁴-corticotropin, useful as an adrenocorticotropic hormone; cyclosporin, a cyclic protein containing 11 amino acids and a 3-hydroxy4-methyl-2-(methylamino)-6-octenoyl moiety at the 6-position, useful as an immunosuppressant; dacliximab (Ro-24-7375), a humanized anti-TAC monoclonal antibody comprised of four subunits linked via disulfide bridges and a molecular weight of approximately 150 kD, useful as an immunosuppressant; daclizumab; daptomycin, a proteinaceous antibacterial agent; desirudin, 63-desulfohirudin from Hirudo medicinalis comprising 63 amino acids, useful as an anticoagulant; deslorelin, luteinizing hormone-releasing factor (pig) comprising 9 amino acids, useful as an LHRH agonist; desmopressin acetate, vasopressin, 1-(3-mercaptopropanoic acid)-8-D-Arg-, monoacetate salt, trihydrate, comprising 9 amino acids, useful as an antidiuretic; detirelix acetate comprising 10 amino acids, useful as an LHRH antagonist; dumorelin, 27-L-Leu-44a-Gly growth hormone-releasing factor (human); elcatonin, 1-butyric acid-7-(L-2-aminobutyric acid)-26-L-Asp-27-L-Val-29-L-Ala calcitonin (salmon); emoctakin, interleukin 8 (human) comprising 72 amino acids with two Cys bridges; epoetin alfa, a 165 amino acid glycoprotein that regulates red blood cell production and is produced by Chinese hamster ovary cells into which the human erythropoietin gene has been inserted, useful as an anti-anemic and hematinic agent; ersofermin, recombinant human basic fibroblast growth factor (bFGF) comprising 157 amino acids, a non-glycosylated protein isolated from human placenta and cloned and expressed in E. coli, useful as a wound healing agent; felypressin is vasopressin, 2-L-Phe-8-L-Lys comprising 9 amino acids, useful as a vasoconstrictor; filgrastim, a single chain 175 amino acid polypeptide, non-glycosylated and expressed by E coli, useful as an antineutropenic agent and as a haematopoietic stimulant; glucagon, a single chain protein of 29 amino acids, useful an antidiabetic agent; gonadorelin acetate, the diacetate salt of luteininzing hormone-releasing factor acetate comprising 10 amino acids, useful as a gonad-stimulating principle; goserelin, luteinizing hormone-releasing factor (pig) comprising 9 amino acids, useful as an LHRH agonist; histrelin, luteinizing hormone-releasing factor (pig) comprising 9 amino acids, useful as an LHRH agonist; imiglucerase, 495-L-Histidineglucosylceramidase placenta isoenzyme protein, useful as an enzyme replenisher for glucocerebrosidase; insulin, dalanated, an insulin derivative prepared by removal of the C-terminal alanine from the B chain of insulin, useful as an antidiabetic agent; interferon alfa-2a, interferon αA (human leukocyte protein moiety reduced) comprising 165 amino acids, useful as an antineoplastic agent and as a biological response modifier; interferon alfa-2b, interferon α2b (human leukocyte clone Hif-SN206 protein moiety reduced) comprising 165 amino acids, also useful as an antineoplastic agent and as a biological response modifier; interferon beta-1a, a glycosylated polypeptide consisting of 166 amino acid residues produced from cultured Chinese hamster ovary cells containing the engineered gene for human interferon beta, also useful as an antineoplastic agent and as a biological response modifier; interferon beta-1b, a non-glycosylated polypeptide consisting of 165 amino acid residues produced from E. coli, also useful as an immunomodulator; interferon gamma-1b, 1-139 interferon γ (human lymphocyte protein moiety reduced), N²-L-Met, useful as an antineoplastic agent and as an immunomodulator; iroplact, N-methionylblood platelet factor 4 (human subunit) comprising 71 amino acid residues having two Cys bridges; lanoteplase, a tissue plasminogen activator protein derived from human t-PA by deletion of the fibronectin-like and the EGF-like domains and mutation of Asn 117 to Gln 117, produced by expression in a mammalian host cell of a DNA sequence encoding the peptide sequence, useful as a plasminogen activator and thrombolytic agent; lanreotide acetate comprising 8 amino acids and one disulfide bridge, useful as an antineoplastic agent; lenograstim, a glycoprotein consisting of 174 amino acid residues produced in Chinese hamster ovary cells by expression of a human granulocyte colony-stimulating factor-cDNA derived from a human oral cavity squamous cell line-mRNA, useful as an antineutropenic agent and as an haematopoietic stimulant; lutrelin acetate, a luteinizing hormone-releasing factor (pig) comprising 9 amino acids, useful as an LHRH agonist; molgramostim, a colony-stimulating factor 2 (human clone pHG₂₅ protein moiety reduced) comprising 127 amino acids, useful as an antineutropenic agent and as an haematopoietic stimulant; murodermin, an epidermal growth factor (mouse salivary gland); nafarelin acetatem, luteinizing hormone-releasing factor (pig) comprising 9 amino acids, useful as an LHRH agonist; nagrestipen, 26-L-Alaninelymphokine MiP 1α (human clone pAT 464 macrophage inflammatory comprising 69 amino acids and having two disulfide bridges; pepstatin, N-(3-methyl-1-oxobutyl)-L-Val-L-Val-4-amino-3-hydroxy-6-methylheptanoyl-L-Ala-4-amino-3-hydroxy-6-methylheptanoic acid, useful as a pepsin enzyme inhibitor; pramlintide, a protein comprising 37 amino acids and having one disulfide bridge, useful as an antidiabetic agent; proinsulin human, proinsulin (pig) comprising 86 amino acid residues and having three disulfide bridges, useful as an antidiabetic agent; sargramostim, colony-stimulating factor 2 (human clone pHG25 protein moiety), 23-L-Leu-, a single chain, glycosylated polypeptide of 127 amino acid residues expressed from Saccharomyces cerevisiae, useful as an antineutropenic agent and a haematopoietic stimulant; naturally occurring and synthetically, including recombinantly derived human and animal somatotropins (growth hormones), especially bovine and porcine somatotropins; somagrebove, somatotropin (ox reduced), 1-[N²-L-Met-L-α-Asp-L-Glutamine]- comprising 191 amino acids, useful as a galactopoietic agent especially for veterinary use; somalapor, somatotropin (pig clone pPGH-1 reduced), N-L-Alanyl-growth hormone comprising a total of 191 amino acids, useful as a hormone (growth, porcine); somatrem, somatotropin (human), N-L-Met- comprising 191 amino acids having two disulfide bridges, useful as a growth hormone; somatotropin, a single polypeptide chain comprising 191 amino acids having the normal structure of the principal growth stimulating hormone obtained from the anterior lobe of the human pituitary gland, useful as a growth hormone; somatotropin, available in recombinant form; somavubove, somatotropin (ox), 127-L-Leu-, one of the four naturally occurring molecular variants in bovine pituitary somatotropin, useful as a galactopoietic agent; somenopor, somatotropin (pig clone pPGH-1 reduced), N-L-Ala-32-de-L-Glu-33-de-L-Arg-34-de-L-Ala-35-de-L-Tyr-36-de-L-Ile-37-de-L-Pro-38-de-L-Glu- comprising 190 amino acids, useful as a porcine growth hormone, sometribove, somatotropin (ox), 1-L-Met-127-L-Leu- comprising 191 amino acids, useful as a veterinary growth stimulant; sometripor, somatotropin (pig recombinant) C₉₇₉H₁₅₂₇N₂₆₅O₂₈₇S₈, somfasepor, somatotropin (pig recombinant) C₉₃₈H₁₄₆₅N₂₅₇O₂₇₈S₆; somidobove, somatotropin (ox recombinant) C₁₀₂₀H₁₅₉₆N₂₇₄O₃₀₂S₉; teprotide, bradykinin potentiator B, 2-L-Trp-3-de-L-Leu-4-de-L-Pro-8-L-Glutamine- comprising 9 amino acids, useful as an angiotensin-converting enzyme inhibitor; teriparatide, a protein comprising 34 amino acids, useful as a bone resorption inhibitor and an osteoporosis therapy adjunct; thymalfasin, thymosin α1 (ox) comprising 28 amino acids, useful as an antineoplastic agent, in treating hepatitis and infectious diseases, and as a vaccine enhancer; thymopentin, a pentapeptide useful as an immunoregulator; triptorelin, luteinizing hormone-releasing factor (pig), 6-D-Trp comprising 10 amino acids, useful as an antineoplastic agent; vapreotide comprises 8 amino acids having one disulfide bridge, useful as an antineoplastic agent; vasopressin in the 8-L-Arg- or 8-L-Lys- form comprising 9 amino acids having one disulfide bridge, useful as an antidiuretic hormone; myoglobin; hemoglobin; p-lactoglobulin; immunoglobulin-G (IgG); antihemophilic factor (Factor VIII); lysozyme; ubiquitin; platelet-activating factor (PAF); tumor necrosis factor-α (TNF-α); tumor necrosis factor-β (TNF-β); macrophage inflammatory protein (MIP); heparin; eosinophil cationic protein (ECP); recombinant factor IX; monoclonal antibody for non-Hodgkin's B-cell lymphoma; interferon alpha, useful for treating hepatitis C; and fibroblast-derived artificial skin for treating wounds and burns.

5. A process according to claim 1 wherein said conditions which are effective to drive said condensation reaction substantially to completion comprise:
(a) conditions which change any water present from the liquid phase to the gaseous or solid phase whereby said water is removed from said aqueous environment of said condensation reaction; and
(b) conditions which optimize the energy input to said process necessary most efficiently to separate said water of said aqueous environment in which the condensation reaction takes place, including water produced by said condensation reaction itself, from the starting material reactants and the condensation adduct final product.

6. A process according to claim 5 wherein at temperatures above 0° C. said conditions which change any water present from the liquid phase to the gaseous phase and which optimize energy input to said process comprise (a) heating said reaction mixture in said aqueous environment to the highest temperature consistent with maintaining the integrity of said protein starting material reactant and said condensation adduct final product, as well as consistent with optimal efficiencies and economies for carrying out said preparation process including said condensation reaction; (b) subdividing said reaction mixture in said aqueous environment into the smallest droplets consistent with maintaining the integrity of said protein starting material reactant and said condensation adduct final product, as well as consistent with optimal efficiencies and economies for carrying out said preparation process including said condensation reaction; and (c) providing said droplets thus formed with the highest comparative velocity, referenced to a gas inert thereto through which they pass, which is consistent with maintaining the integrity of said protein starting material reactant and said condensation adduct final product, as well as consistent with optimal efficiencies and economies for carrying out said preparation process including said condensation reaction.

7. A process according to claim 6 wherein said reaction mixture is heated to a temperature of from 25° C. to 125° C., while maintaining said aqueous environment in the liquid phase by the application of elevated pressure where necessary.

8. A process according to claim 7 wherein said reaction mixture is heated to a temperature of from 75° C. to 105° C.

9. A process according to claim 6 wherein said reaction mixture in said aqueous environment is divided into droplets having an average diameter of from 1.0 μm to 5.0 mm.

10. A process according to claim 6 wherein said comparative velocity to which said droplets are subjected is from 0.1 m/sec to 5.0 m/sec.

11. A process according to claim 6 wherein said subdividing of said reaction mixture into droplets is accomplished using a spraying apparatus comprising any suitable combination of high pressure gas stream generators and associated dispersing means together with high pressure hydraulic pumping means and associated nozzle means.

12. A process according to claim 6 wherein said subdividing of said reaction mixture into droplets is accomplished using mechanical action in the form of a rapidly rotating disc over the surface of which an aqueous stream comprising each said starting material reactant is directed, and said aqueous stream traverses said disc in such manner that it is propelled from the edge of said disc in droplet form, and which takes place under conditions adjusted with regard to temperature, humidity and pressure so as eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

13. A process according to claim 6 carried out under conditions of reduced moisture whereby the rate of water removal is accelerated and the overall amount removed is increased.

14. A process according to claim 13 wherein additional amounts of moisture are removed so that the amount of moisture present is as low as from 0.1% to 0.001% by weight, based on the weight of the final product.

15. A process according to claim 6 carried out under conditions such that higher amounts of moisture are present in the final product in order to maintain the integrity thereof, wherein said higher amounts of moisture are in the range of from 3.0% to 20.0% by weight.

16. A process according to claim 5 wherein at temperatures of 0° C. and below said conditions which change any water present from the liquid phase to the solid phase and which optimize energy input to said process comprise (a) cooling said reaction mixture in said aqueous environment to a temperature sufficiently low to freeze substantially all of the unbound liquid water present in said aqueous environment, said temperature being consistent with maintaining the integrity of the protein starting material reactant and the condensation adduct final product, as well as consistent with optimal efficiencies and economies for carrying out said preparation process including said condensation reaction; (b) subjecting said thus cooled reaction mixture in said frozen aqueous environment to a reduced pressure in the presence of a gas inert thereto, which is consistent with maintaining the integrity of said protein starting material reactant and said condensation adduct final product, as well as consistent with optimal efficiencies and economies for carrying out said preparation process including said condensation reaction.

17. A process according to claim 16 wherein said reaction mixture is cooled to a temperature of from −110° C. to 0° C.

18. A process according to claim 16 wherein said reduced pressure to which said cooled reaction mixture in said frozen aqueous environment is subjected is from 5.0 mmHg absolute to 0.0001 mmHg absolute.

19. A process according to claim 16 carried out under conditions of reduced moisture whereby the rate of water removal is accelerated and the overall amount removed is increased.

20. A process according to claim 16 wherein additional amounts of moisture are removed so that the amount of moisture present is as low as from 0.1% to 0.001% by weight, based on the weight of the final product.

21. A process according to claim 16 carried out under conditions such that higher amounts of moisture are present in the final product in order to maintain the integrity thereof, wherein said higher amounts of moisture are in the range of from 3.0% to 20.0% by weight.

22. A Schiff base condensation adduct final product comprising a protein and an aromatic o-hydroxy aldehyde prepared under conditions effective to eliminate from about 97.0% to about 99.9% by weight, preferably from about 98.0% to about 99.0% by weight of the water already present or produced during said condensation reaction, consistent with maintaining the integrity of the condensation reactants and adduct final product, and to assure a rate of conversion to said condensation adduct final product, i.e., with resulting yield of said condensation adduct final product of equal to or greater than about 98.5% by weight, preferably equal to or greater than about 99.5% by weight based on the weight of the reactants.

23. A product according to claim 22 in droplet form wherein said droplets have mean diameters in the range of from about 0.1 μm to about 10.0 μm.

24. A product according to claim 22 wherein said aromatic o-hydroxy aldehyde comprises one or more compounds of the formula: 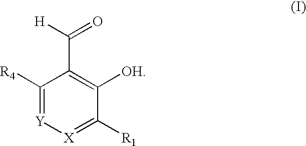
wherein:
R₁ and R₄ are independently selected from the group consisting essentially of hydrogen; hydroxy; halo; nitro; cyano; trifluoromethyl; (C₁-C₆)alkyl; (C₁-C₆)alkoxy; (C₃-C₆)cycloalkyl; (C₂-C₆)alkenyl; —C(═O)OR₇ —OC(═O)R₇; —S(═O)₂—S(═O)₂N(R₇)(R₉); —S(═O)₂R₇; —S(═O)₂OR₇; —C(═O)NR₇R₉; —C(═O)R₉; and —N(R₇)(R₉), where R₇ is hydrogen or (C₁-C₄)alkyl and R₉ is (C₁-C₄) alkyl;
wherein:
said alkyl, cycloalkyl and alkenyl groups defining R₁ and R₄ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl, carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl;
X and Y are independently N, or CHR₂ or CHR₃, respectively, where R₂ and R₃ are independently selected from the group consisting essentially of hydrogen; hydroxy; halo; nitro, cyano; trifluoromethyl; (C₁-C₆)alkyl; (C₁-C₆)alkoxy, (C₃-C₆)cycloalkyl; (C₂-C₆)alkenyl, —C(═O)OR₁₁; —OC(═O)R₁₁; —S(═O)₂, —S(═O)₂N(R₁₁)(R₁₃); and —N(R₁₁)(R₁₃), where R₁₁ is hydrogen or (C₁-C₄)alkyl and R₁₃ is (C₁-C₄)alkyl; and wherein said alkyl, cycloalkyl and alkenyl groups defining R₂ and R₃ may optionally be independently substituted by one or two substituents selected from the group consisting essentially of halo; hydroxy; (C₁-C₂)alkyl; (C₁-C₂)alkoxy; (C₁-C₂)alkoxy-(C₁-C₂)alkyl; (C₁-C₂)alkoxycarbonyl; carboxyl; (C₁-C₂)alkylcarbonyloxy; nitro; cyano; amino disubstituted by (C₁-C₂)alkyl; sulfonyl; and sulfonamido disubstituted by (C₁-C₂)alkyl.

25. A product according to claim 24 wherein said aromatic o-hydroxy aldehyde comprises o-vanillin; salicylaldehyde; 2,3-dihydroxybenzaldehyde; 2,6-dihydroxybenzaldehyde; 2-hydroxy-3-ethoxybenzaldehyde; or pyridoxal.

26. A product according to claim 24 wherein said protein having beneficial activity in animals comprises one or more members selected from the group consisting of:
proteinaceous endogenous and synthetic opioid analgesics and antagonists comprising enkephalins, endorphins, and dynorphins which are selective and nonselective agonists and antagonists of the μ, κ, and δ opioid receptor subtypes, including [Leu⁵] and [Met⁵]enkephalin; dynorphin A and B; α- and β-neoendorphin; [D-Ala², MePhe⁴, -Gly(ol)⁵]enkephalin (DAMGO); [D-Pen², D-Pen⁵]enkephalin (DPDPE); [D-Ser², Leu⁵]enkephalin-Thr⁶ (DSLET); [D-Ala², D-Leu⁵]enkephalin (DADL); D-Phe-Cys-Tyr-D-Trp-Orn-Thr-Pen-Thr-NH₂ (CTOP); [D-Ala², N-MePhe⁴, Met(O)⁵-ol]enkephalin (FK-33824); Tyr-D-Ala-Phe-Asp-Val-Val-Gly-NH₂ ([D-Ala²]deltorphin 1; Tyr-D-Ala-Phe-Glu-Val-Val-Gly-NH₂ ([D-Ala², Glu⁴]deltorphin II; Tyr-Pro-Phe-Pro-NH₂ (morphiceptin); Tyr-Pro-MePhe-D-Pro-NH₂ (PL-017); and [D-Ala², Leu⁵, Cys6]enkephalin;
autocoids including bradykinin and kallidin produced by proteolytic reactions in response to inflammatory events selected from tissue damage, viral infections, and allergic reactions, wherein said proteins act locally to produce pain, vasodilatation, increased vascular permeability and the synthesis of prostaglandins, wherein said proteins have agonist and antagonist activity and are useful for the treatment of male infertility, for the delivery of cancer chemotherapeutic agents beyond the blood-brain barrier, and for the treatment of pain, asthma, and other chronic inflammatory diseases, including: Arg-Pro-Pro-Gly-Phe-Ser-Pro-Phe-Arg (bradykinin); Lys-Arg-Pro-Pro-Gly-Phe-Ser-Pro-Phe-Arg (kallidin); Arg-Pro-Pro-Gly-Phe-Ser-Pro-Phe (des-Arg⁹-bradykinin); Lys-Arg-Pro-Pro-Gly-Phe-Ser-Pro-Phe (des-Arg¹⁰-kallidin); Arg-Pro-Pro-Gly-Phe-Ser-Pro-Leu (des-Arg⁹-[Leu⁸]-bradykinin); Arg-Pro-Pro-Gly-Phe-Ser-[D-Phe]-Phe-Arg ([D-Phe⁷]-bradykinin); and [D-Arg]-Arg-Pro-Hyp-Gly-Thi-Ser-Tic-Oic-Arg (HOE 140), where Hyp is trans4-hydroxy-Pro; Thi is β-(2-thienyl)-Ala; Tic is [D]-1,2,3,4-tetrahydroquinolin-3-yl-carbonyl; and Oic is (3 as, 7 as)-octahydroindol-2-yl-carbonyl;
proteins active at vasopressin receptor subtypes V₁ and V₂ which mediate pressor responses and antidiuretic responses, respectively, including V₁ antagonists beneficial in the treatment of congestive heart failure, hypertension, and postoperative ileus and abdominal distension, V₂ agonists used to treat central diabetes insipidus by controlling polyuria and polydipsia, and to treat bleeding disorders including von Willebrand's disease, including the specific naturally-occurring vasopressin-like peptides: arginine vasopressin (AVP) of the following formula: 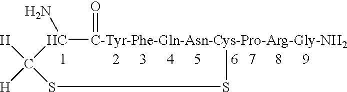
and lypressin ([Lys⁸]-AVP; synthetic vasopressin peptides: V₁ₐ-selective agonist [Phe², Ile², Orn⁸]AVP; V_{1b}-selective agonist deamino [D-3-(3′-pyridyl)-Ala²]AVP; V₂-selective agonists desmopressin (dDAVP), and deamino[Val⁴, D-Arg⁸]AVP; and peptide antagonists including V₁ₐ-selective antagonist d(CH₂)₅[Tyr(Me)²]AVP of the formula: 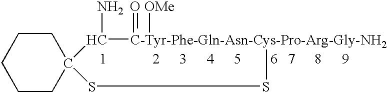
and V_{1b}-selective antagonist dp[Tyr(Me)²]AVP; and V₂-selective antagonists des Gly-NH₂⁹-d(CH₂)₅[D-Ile², Ile⁴]AVP, and d(CH₂)₅[D-Ile², Ile⁴, Ala-N H₂⁹]AVP;
pentagastrin used as an indicator of gastric secretion of the formula: N-t-butyloxycarbonyl-β-Ala-Trp-Met-Asp-Phe-NH₂;
octreotide useful in treating the symptoms of tumors of the gastrointestinal tract, diarrhea refractory to other treatment, motility disorders, and gastrointestinal bleeding of the formula: L-cysteinamide-D-Phe-L-Cys-L-Phe-D-Trp-L-Lys-L-Thr-N-[2-hydroxy-1-(hydroxymethyl)propyl]-. cyclic (2→7)-disulfide, [R—(R*,R*)]—;
antibody reagents useful as immunosuppressive agents including antithymocyte globulin; muromonab-CD3 monoclonal antibody; and Rh₀(D) immune globulin; and protein immunostimulants useful in treating immunodeficiency states, including immune globulin;
cytokines produced by leukocytes and having a variety of immunoregulatory effects, including: interferons, colony-stimulating factors, and interleukins, and specifically α-interferon; interferon-γ (IFN-γ); granulocyte colony-stimulating factor (G-CSF); granulocyte macrophage colony-stimulating factor (GM-CSF); and interleukin-1 (IL-1) through interleukin-12 (IL-12);
hematopoietic growth factors involved in the regulation of the process whereby mature blood cells are continuously replaced, useful in the treatment of primary hematological diseases and uses as adjunctive agents in the treatment of severe infections and in the management of patients who are undergoing chemotherapy or marrow transplantation, including specifically: growth factors including erythropoietin (EPO); stem cell factor (SCF), interleukins (IL-1-12); monocyte/macrophage colony-stimulating factor (M-CSF, CSF-1); P1XY321 (GM-CSF/IL-3 fusion protein); and thrombopoietin;
thrombolytic proteins useful for dissolving both pathological thrombi and fibrin deposits at sites of vascular injury, including streptokinase; tissue plasminogen activator (t-PA); and urokinase;
anterior pituitary hormones and the hypothalamic factors that regulate their use comprising: (a) somatotropic hormones including growth hormone (GH), prolactin (Prl), and placental lactogen (PL); (b) glycoprotein hormones including luteinizing hormone (LH), follicle-stimulating hormone (FSH), and thyroid-stimulating hormone (TSH); and (c) POMC-derived hormones including corticotropin (ACTH), α-melanocyte-stimulating hormone (α-MSH), β-melanocyte-stimulating hormone (β-MSH), β-lipotropin (β-LPH), and γ-lipotropin (γ-LPH); the hypothalamic factors regulating release of said hormones, including growth hormone-releasing hormone (GHRH), luteininzing hormone releasing hormone (LHRH), insulin-like growth factor (IGF-1 and IGF-2), somatostatin, and gonadotropin-releasing hormone (GnRH);
growth hormone useful as replacement therapy in growth-hormone deficient children, including: somatostatin, the synthetic analogue of somatostatin, octreotide; gonadotropic hormones including LH, FSH, and corionic gonadotropin (GC) useful in the diagnoses of reproductive disorders and in the treatment of infertility, including: urofollitropin, a human menopausal gonadotropin (hMG) from which substantially most of the LH has been removed useful for inducing ovulation, and gonadorelin, a synthetic human GnRH useful for stimulating gonadotropin secretion; synthetic GnRH agonists including: leuprolide, histrelin, nafarelin, and goserelin useful in treating endocrine disorders that are responsive to reductions in gonadal steroids;
thyrotropin (TSH), the secretion of which is controlled by thyrotropin-releasing hormone (TRH), useful for hormone replacement therapy in patients with hypothyroidism and for TSH suppression therapy in patients with nontoxic goiter or after treatment for thyroid cancer;
insulin for treating insulin-dependent diabetes mellitus patients and non-insulin-dependent diabetes mellitus patients; glucagon which has a physiological role in the regulation of glucose and ketone body metabolism, useful in treating severe hypoglycemia, and by radiologists for inhibiting the gastrointestinal tract; somatostatin, useful for blocking hormone release in endocrine-secreting tumors, including insulinomas, glucagonomas, VIPomas, carcinoid tumors, and somatotropinomas, and the synthetic analogue, octreotide;
calcitonin, a hormone acting specifically on osteoclasts to inhibit bone resorption, is useful in managing hypercalcemia and in disorders of increased skeletal remodeling, including Paget's disease; parathyroid hormone, useful in the treatment of patients with spinal osteoporosis;
aldesleukin, 125-L-serine-2-133-interleukin 2, useful as an antineoplastic agent and as an immunostimulant; alglucerase, a monomeric glycoprotein of 497 amino acids and a modified form of human placental tissue β-glucocerebrosidase, is useful as a replenisher of the glucocerebrosidase enzyme; alsactide, a synthetic corticotropin analogue: 1-β-Ala-17 [L-2,6-diamino-N-(4-aminobutyl)hexanamide]-α¹⁻¹⁷-corticotropin; alteplase, a serine protease of 527 amino acids whose sequence is identical to the naturally occurring protease produced by endothelial cells in vessel walls, useful as a plasminogen activator; alvircept sudotox, a synthetic chimeric protein engineered to link the first 178 amino acids of the extracellular domain of CD₄ via two linker residues to amino acids 1-3 and 253-613 of Pseudomonas exotoxin A, useful as an antiviral agent; amlintide, a protein of 37 amino acids, useful as an antidiabetic agent; amogastrin: N-carboxy-L-Trp-L-Met-L-α-Asp-3-phenyl-L-Alaninamide; anakinra: N²-L-Met-interleukin 1 receptor antagonist useful as a nonsteroidal anti-inflammatory and as a suppressant for treating inflammatory bowel disease; anaratide acetate, atriopeptin-21 (rat), N-L-Arg-8-L-Met-21a-L-Phe-21b-L-Arg-21c-L-Tyr-, acetate, useful as an antihypertensive agent and as a diuretic; angiotensin amide, angiotensin II, 1-L-Asn-5-L-Val-, useful as a vasoconstrictor; aprotinin, a pancreatic trypsin inhibitor having 58 amino acids, useful as an enzyme inhibitor (proteinase); arfalasin, 1-succinamic acid-5-L-Val-8-(L-2-phenylglycine)angiotensin II, useful as an antihypertensive agent; argipressin tannate, vasopressin, 8-L-Arg-, tannate, useful as an antidiuretic; aspartocin, oxytocin, 4-L-Asn-, is useful as an antibiotic agent produced by Streptomyces griseus; atosiban, oxytocin, 1-(3-mercaptopropanoic acid)-2-(O-ethyl-D-Tyr)-4-L-Thr-8-L-Orn-, useful as an oxytocin antagonist; avoparcin, a glycopeptide antibiotic obtained from Streptomyces candidus; basifungin, N-[(2R,3R)-2-hydroxy-3-MeVal]-N-L-MeVal-L-Phe-N-L-MePhe-L-Pro-L-allo-Ile-N-L-MeVal-L-Leu-3-hydroxy-N-L-MeVal α₁-lactone, useful as an antifungal agent; becaplermin, recombinant human platelet-derived growth factor B, a recombinant protein produced by genetically engineered Saccharomyces cerevisiae similar in amino acid composition and biological activity to endogenous human PDGF-BB homodimer, useful for treating chronic dermal ulcers by promoting proliferation of mesenchymally-derived cells; bivalirudin, an anticoagulant, antithrombotic agent having 20 amino acids; carbetocin, 1-butyric acid-2-[3-(p-methoxyphenyl)-L-Ala]oxytocin; cargutocin, 1-butyric acid-6-(L-2-aminobutyric acid)-7-glycineoxytocin; ceruletide, 5-O-L-Pro-L-Gln-L-α-Asp-L-O-sulfo-L-Tyr-L-Thr-L-Gly-L-Trp-L-Met-L-α-Asp-L-Phe-amide, useful as a gastric secretory stimulant; cetermin, transforming human growth factor β2 having 112 amino acids; cilmostim, 1-233-colony-stimulating factor 1 (human clone p3ACSF-69 protein moiety), cyclic (7→90), (48→139), (102→146)-tris(disulfide) dimer, useful as a hematopoietic agent (macrophage colony-stimulating factor); colistimethate sodium, a colistin A component useful as an antibacterial agent; corticorelin, ovine triflutate, corticotropin-releasing factor (sheep), trifluoroacetate salt, useful as a diagnostic aid for adrenocortical insufficiency and Cushing's syndrome, and as a corticotropin-releasing hormone; cosyntropin, tetracosactide acetate, α¹⁻²⁴-corticotropin, useful as an adrenocorticotropic hormone; cyclosporin, a cyclic protein containing 11 amino acids and a 3-hydroxy-4-methyl-2-(methylamino)-6-octenoyl moiety at the 6-position, useful as an immunosuppressant; dacliximab (Ro-24-7375), a humanized anti-TAC monoclonal antibody comprised of four subunits linked via disulfide bridges and a molecular weight of approximately 150 kD, useful as an immunosuppressant; daclizumab; daptomycin, a proteinaceous antibacterial agent; desirudin, 63-desulfohirudin from Hirudo medicinalis comprising 63 amino acids, useful as an anticoagulant; deslorelin, luteinizing hormone-releasing factor (pig) comprising 9 amino acids, useful as an LHRH agonist; desmopressin acetate, vasopressin, 1-(3-mercaptopropanoic acid)-8-D-Arg-, monoacetate salt, trihydrate, comprising 9 amino acids, useful as an antidiuretic; detirelix acetate comprising 10 amino acids, useful as an LHRH antagonist; dumorelin, 27-L-Leu-44a-Gly growth hormone-releasing factor (human); elcatonin, 1-butyric acid-7-(L-2-aminobutyric acid)-26-L-Asp-27-L-Val-29-L-Ala calcitonin (salmon); emoctakin, interleukin 8 (human) comprising 72 amino acids with two Cys bridges; epoetin alfa, a 165 amino acid glycoprotein that regulates red blood cell production and is produced by Chinese hamster ovary cells into which the human erythropoietin gene has been inserted, useful as an anti-anemic and hematinic agent; ersofermin, recombinant human basic fibroblast growth factor (bFGF) comprising 157 amino acids, a non-glycosylated protein isolated from human placenta and cloned and expressed in E. coli, useful as a wound healing agent; felypressin is vasopressin, 2-L-Phe-8-L-Lys comprising 9 amino acids, useful as a vasoconstrictor; filgrastim, a single chain 175 amino acid polypeptide, non-glycosylated and expressed by E coli, useful as an antineutropenic agent and as a haematopoietic stimulant; glucagon, a single chain protein of 29 amino acids, useful an antidiabetic agent; gonadorelin acetate, the diacetate salt of luteininzing hormone-releasing factor acetate comprising 10 amino acids, useful as a gonad-stimulating principle; goserelin, luteinizing hormone-releasing factor (pig) comprising 9 amino acids, useful as an LHRH agonist; histrelin, luteinizing hormone-releasing factor (pig) comprising 9 amino acids, useful as an LHRH agonist; imiglucerase, 495-L-Histidineglucosylceramidase placenta isoenzyme protein, useful as an enzyme replenisher for glucocerebrosidase; insulin, dalanated, an insulin derivative prepared by removal of the C-terminal alanine from the B chain of insulin, useful as an antidiabetic agent; interferon alfa-2a, interferon αA (human leukocyte protein moiety reduced) comprising 165 amino acids, useful as an antineoplastic agent and as a biological response modifier; interferon alfa-2b, interferon α2b (human leukocyte clone Hif-SN206 protein moiety reduced) comprising 165 amino acids, also useful as an antineoplastic agent and as a biological response modifier; interferon beta-1a, a glycosylated polypeptide consisting of 166 amino acid residues produced from cultured Chinese hamster ovary cells containing the engineered gene for human interferon beta, also useful as an antineoplastic agent and as a biological response modifier; interferon beta-1b, a non-glycosylated polypeptide consisting of 165 amino acid residues produced from E. coli, also useful as an immunomodulator; interferon gamma-1b, 1-139 interferon γ (human lymphocyte protein moiety reduced), N²-L-Met, useful as an antineoplastic agent and as an immunomodulator; iroplact, N-methionylblood platelet factor 4 (human subunit) comprising 71 amino acid residues having two Cys bridges; lanoteplase, a tissue plasminogen activator protein derived from human t-PA by deletion of the fibronectin-like and the EGF-like domains and mutation of Asn 117 to Gln 117, produced by expression in a mammalian host cell of a DNA sequence encoding the peptide sequence, useful as a plasminogen activator and thrombolytic agent; lanreotide acetate comprising 8 amino acids and one disulfide bridge, useful as an antineoplastic agent; lenograstim, a glycoprotein consisting of 174 amino acid residues produced in Chinese hamster ovary cells by expression of a human granulocyte colony-stimulating factor-cDNA derived from a human oral cavity squamous cell line-mRNA, useful as an antineutropenic agent and as an haematopoietic stimulant; lutrelin acetate, a luteinizing hormone-releasing factor (pig) comprising 9 amino acids, useful as an LHRH agonist; molgramostim, a colony-stimulating factor 2 (human clone pHG₂₅ protein moiety reduced) comprising 127 amino acids, useful as an antineutropenic agent and as an haematopoietic stimulant; murodermin, an epidermal growth factor (mouse salivary gland); nafarelin acetatem, luteinizing hormone-releasing factor (pig) comprising 9 amino acids, useful as an LHRH agonist; nagrestipen, 26-L-Alaninelymphokine MiP 1α (human clone pAT 464 macrophage inflammatory comprising 69 amino acids and having two disulfide bridges; pepstatin, N-(3-methyl-1-oxobutyl)-L-Val-L-Val-4-amino-3-hydroxy6-methylheptanoyl-L-Ala-4-amino-3-hydroxy-6-methylheptanoic acid, useful as a pepsin enzyme inhibitor; pramlintide, a protein comprising 37 amino acids and having one disulfide bridge, useful as an antidiabetic agent; proinsulin human, proinsulin (pig) comprising 86 amino acid residues and having three disulfide bridges, useful as an antidiabetic agent; sargramostim, colony-stimulating factor 2 (human clone pHG25 protein moiety), 23-L-Leu-, a single chain, glycosylated polypeptide of 127 amino acid residues expressed from Saccharomyces cerevisiae, useful as an antineutropenic agent and a haematopoietic stimulant; naturally occurring and synthetically, including recombinantly derived human and animal somatotropins (growth hormones), especially bovine and porcine somatotropins; somagrebove, somatotropin (ox reduced), 1-[N²-L-Met-L-α-Asp-L-Glutamine]- comprising 191 amino acids, useful as a galactopoietic agent especially for veterinary use; somalapor, somatotropin (pig clone pPGH-1 reduced), N-L-Alanyl-growth hormone comprising a total of 191 amino acids, useful as a hormone (growth, porcine); somatrem, somatotropin (human), N-L-Met- comprising 191 amino acids having two disulfide bridges, useful as a growth hormone; somatotropin, a single polypeptide chain comprising 191 amino acids having the normal structure of the principal growth stimulating hormone obtained from the anterior lobe of the human pituitary gland, useful as a growth hormone; somatotropin, available in recombinant form; somavubove, somatotropin (ox), 127-L-Leu-, one of the four naturally occurring molecular variants in bovine pituitary somatotropin, useful as a galactopoietic agent; somenopor, somatotropin (pig clone pPGH-1 reduced), N-L-Ala-32-de-L-Glu-33-de-L-Arg-34-de-L-Ala-35-de-L-Tyr-36-de-L-Ile-37-de-L-Pro-38-de-L-Glu- comprising 190 amino acids, useful as a porcine growth hormone; sometribove, somatotropin (ox), 1-L-Met-127-L-Leu- comprising 191 amino acids, useful as a veterinary growth stimulant; sometripor, somatotropin (pig recombinant) C₉₇₉H₁₅₂₇N₂₆₅O₂₈₇S₈; somfasepor, somatotropin (pig recombinant) C₉₃₈H₁₄₆₅N₂₅₇O₂₇₈S₆; somidobove, somatotropin (ox recombinant) C₁₀₂₀H₁₅₉₆N₂₇₄O₃₀₂S₉; teprotide, bradykinin potentiator B, 2-L-Trp-3-de-L-Leu-4-de-L-Pro-8-L-Glutamine- comprising 9 amino acids, useful as an angiotensin-converting enzyme inhibitor; teriparatide, a protein comprising 34 amino acids, useful as a bone resorption inhibitor and an osteoporosis therapy adjunct; thymalfasin, thymosin α1 (ox) comprising 28 amino acids, useful as an antineoplastic agent, in treating hepatitis and infectious diseases, and as a vaccine enhancer; thymopentin, a pentapeptide useful as an immunoregulator; triptorelin, luteinizing hormone-releasing factor (pig), 6-D-Trp comprising 10 amino acids, useful as an antineoplastic agent; vapreotide comprises 8 amino acids having one disulfide bridge, useful as an antineoplastic agent; vasopressin in the 8-L-Arg- or 8-L-Lys- form comprising 9 amino acids having one disulfide bridge, useful as an antidiuretic hormone; myoglobin; hemoglobin; β-lactoglobulin; immunoglobulin-G (IgG); antihemophilic factor (Factor VIII); lysozyme; ubiquitin; platelet-activating factor (PAF); tumor necrosis factor-α (TNF-α); tumor necrosis factor-β (TNF-β); macrophage inflammatory protein (MIP); heparin; eosinophil cationic protein (ECP); recombinant factor IX; monoclonal antibody for non-Hodgkin's B-cell lymphoma; interferon alpha, useful for treating hepatitis C; and fibroblast-derived artificial skin for treating wounds and burns.

27. A process according to claim 4 in which said protein having beneficial activity in animals comprises a member selected from the group consisting essentially of naturally occurring porcine somatotropin; somalapor, somatotropin (pig clone pPGH-1 reduced), N-L-Alanyl-growth hormone comprising a total of 191 amino acids; somenopor, somatotropin (pig clone pPGH-1 reduced), N-L-Ala-32-de-L-Glu-33-de-L-Arg-34-de-L-Ala-35-de-L-Tyr-36-de-L-Ile-37-de-L-Pro-38-de-L-Glu- comprising 190 amino acids; somatotropin (pig recombinant) C₉₇₉H₁₅₂₇N₂₆₅O₂₈₇S₈; and somfasepor, somatotropin (pig recombinant) C₉₃₈H₁₄₆₅N₂₅₇O₂₇₈S₆.

28. A product according to claim 26 in which said protein having beneficial activity in animals comprises a member selected from the group consisting essentially of naturally occurring porcine somatotropin; somalapor, somatotropin (pig clone pPGH-1 reduced), N-L-Alanyl-growth hormone comprising a total of 191 amino acids; somenopor, somatotropin (pig clone pPGH-1 reduced), N-L-Ala-32-de-L-Glu-33-de-L-Arg-34-de-L-Ala-35-de-L-Tyr-36-de-L-Ile-37-de-L-Pro-38-de-L-Glu- comprising 190 amino acids; somatotropin (pig recombinant) C₉₇₉H₁₅₂₇N₂₆₅O₂₈₇S₈; and somfasepor, somatotropin (pig recombinant) C₉₃₈H₁₄₆₅N₂₅₇O₂₇₈S₆.
